(19) United States
(12) Patent Application Publication
(10) Pub. No.: US 2002/0052000 A1
(43) Pub. Date: May 2, 2002

(54) Diagnostics for and mediators of inflammatory disorders

(76) Inventors: Sampath Parthasarathy, Dunwoody, GA (US); Russell M. Medford, Atlanta, GA (US); R. Wayne Alexander, Atlanta, GA (US)

Correspondence Address: Sherry M. Knowles, Esq., KING & SPALDING, 45th Floor, 191 Peachtree Street, N.E., Atlanta, GA, 30303, US

(21) Appl. No.: 09/779,099

(22) Filed: Feb. 7, 2001

Related U.S. Application Data
(63) Continuation of application No. 08/934,392, filed on Sep. 19, 1997, now abandoned.
(60) Provisional application No. 60/026,401, filed on Sep. 20, 1996.
(60) Provisional application No. 60/039,333, filed on Mar. 17, 1997.

(51) Int. Cl. G01N 33/53

(52) U.S. Cl. 435/7.1

(57) ABSTRACT

A method and kit for the diagnosis and quantification of the state of oxidation, and more specifically, the level of lipid peroxidation, of a host is provided that includes contacting a host biological sample with an antibody to an antigen formed by the reaction of a lipid hydroperoxide with a primary amine. This method assesses the risk of, or existence of, oxidative damage in the host. The invention also includes monoclonal and polyclonal antibodies, as well as antibody fragments, optionally in purified or isolated form, which are useful in this method and kit.

This application claims priority to U.S. Ser. No. 60/026,401, entitled “Diagnostics for and Mediators of Inflammatory Disorders,” filed by Sam Parthasarathy, Russell M. Medford, and Wayne R. Alexander on Sep. 20, 1996 and U.S. Ser. No. 60/039,333, also entitled “Diagnostics for and Mediators of Inflammatory Disorders,” filed by Sam Parthasarathy, Russell Medford, and Wayne R. Alexander, on Mar. 17, 1997.

This application is in the area of methods for the diagnosis and treatment of inflammatory disorders, including cardiovascular disorders.

Oxidative stress has been implicated in a number of diseases. A product of oxidative stress that appears to mediate oxidation-induced disease is peroxidized lipid. The formation of lipid peroxides in biological systems is a complex process that can be brought about by a variety of means, including by enzymes (such as lipoxygenases, cyclooxygenases, peroxidases, and other oxygenases)as well as by non-enzymatic means through the generation of intracellular, extracellular, or cell surface reactive oxygen species. Oxidized lipids of the cell membrane and the extra-cellular milieu can induce profound alterations in cell behavior. The deleterious effects of peroxidized lipids and their degradation products have been well recognized in the pathogenesis of atherosclerosis (Herttuala,et al. Journal of Clinical Investigation, 84:1086-1095 (1989); Gonen,et al., Atherosclerosis, 65:265-272 (1987); and Jurgens,et al., Biochim. Biophys Acta, 875:104-114 (1986)). Oxidation has now been deemed a potential risk factor for cardiovascular diseases (Palinski,et al., Arteriosclerosis, 10:325-335 (1990)).

Since oxidation of lipids is associated with inflammatory and cardiovascular disease states, it is an important medical goal to have a reliable test to quantify and assess the degree of oxidation of lipids in a host, and in particular, a human.

Quantification of lipid hydroperoxides in a host can be based on a direct measurement of the hydroperoxide or on a degradation or reaction product of the hydroperoxide. Polyunsaturated fatty acids (“PUFAs”) have at least two double bounds, and 15-20% have three or more double bonds. Naturally occurring unsaturated fatty acids are never conjugated; the double bonds are separated by at least one methylene group. During oxidation processes into lipid hydroperoxides, the double bonds of PUFAs can become conjugated, which is one of the primary damaging oxidation pathways. The initial products of the oxidation of PUFAs appear to be lipid hydroperoxides (LOOH) and lipid hydroperoxide free radicals (LOOH. or LOO.), most of which have been altered to contain conjugated double bonds. These products, or their further reaction or degradation products, have been used to assay the extent of lipid oxidation in a biological sample.

For example, LOOH can be reduced in vivo to an alcohol, LOH, which is inert. LOH can be detected by any method which assays for alcohols, and if the LOH is conjugated, any method which measures double bond conjugation. Neither of these tests are very specific, however, because in a biological sample, there may be any number of alcohols or conjugated double bond-containing molecules.

Alternatively, LOOH can be oxidized by a metal in vivo to form a radical at the double bond (LOOH.), which is a very reactive species. This molecule often decomposes at the location of oxidation to form two aldehyde containing fragments, or, if it doesn't decompose, it often forms a ketone. The molecule can break down into unsymmetric parts, and if the LOOH has more than two double bonds, it can form a variety of products. If the LOOH has three or more double bonds, malondialdehyde (MDA) is one of the decomposition products. The presence of malondialdehyde can be assayed with thiobarbituric acid (TBA), which reacts with MDA to produce a fluorescent compound that can be easily measured and quantified. TBA reactive substances are referred to as “TBARS.” The TBA test is not appropriate for use as a diagnostic test for the state of a host's lipid oxidation because it is prone to false positives with other aldehydes, sugars, and amino acids. It is useful only in a research setting using carefully controlled samples. Further, it only measures the amount of lipid peroxides that have three or more double bonds.

Another means to measure LOOH in a sample is to react it directly with potassium iodide to form a colored complex, (I₂)KI, which can be easily measured and quantified. A refinement of this test uses leukomethylene blue, which reacts with LOOH to form a colored product. This assay is sold commercially by Kamiya Biomedical Company. Like the TBA method, this method is useful only to assay laboratory samples and cannot be used to assay biological fluids or cell cultures, which can contain numerous cross-reactive substances.

Aldehydes generated during lipid oxidation react with body fluids and tissues. The aldehydes readily modify protein thiols, lysine, histidine, and other residues (Jurgens, et al., Biochemical Journal 265:605-608 (1990); Jessup, Biochemical Journal 234:245-248 (1986); Steinbrecher, et al., J. Lipid Res. 25:1109-1116 (1984)). Such aldehyde modified proteins are antigenic. The antibodies to such epitopes have provided a powerful tool for the detection and localization of aldehyde-modified proteins in the atherosclerotic artery (Boyd, Am. J. Pathol. 135:815-825 (1989); Haberland, et al., Science 241:215-218 (1988); Jurgens, Atheroscler. Throm. 13:1689-1699 (1993)). However, in normal or even atherosclerotic patients, aldehyde-modified protein antigens cannot typically be found in the plasma, but only in low levels in tissue.

Since lipid hydroperoxides do not exist in vivo for a long time because they are inherently unstable, and resulting aldehydes are only one of a number of metabolic routes of decomposition of LOOH and take time to produce, reaction products of the transient LOOH itself with nearby compounds may be present in higher quantities than aldehydes and may represent an appealing diagnostic target. For example, lipid peroxides are highly reactive with amino groups of proteins, lipids and lipophilic molecules. Their close proximity to membrane proteins and apoproteins of lipoproteins may suggest that these modifications may be more relevant than aldehyde induced modifications in biological systems. The generation of LOOH on a cell membrane or lipoprotein is thus more likely to generate, at least in initial stages, proteins that are modified directly by LOOH than proteins that are modified by their extensive degradation products, such as aldehydes. Fruebis, Parthasarathy, and Steinberg, in 1992, published evidence to suggest that a concerted reaction occurs between lipid peroxy radicals and free amino groups of polypeptides or phosphatidylethanolamine to produce fluorescent adducts of unknown structure. Proc. Natl. Acad. Sci. USA, 89: 10588-10592 (1992). Fruebis, et al., incubated linoleoyl hydroperoxide with polypeptides, or an unsaturated phosphatidylethanolamine alone, in the absence of metal ions. The generation of resulting fluorescent product was many times greater than that generated by the incubation of polypeptides or phosphatidylethanolamine with an aldehyde, 4-hydroxynonenal (which produces fluorescent Schiff bases). Fruebis, et al., suggested two possible reaction pathways, both initiated by the interaction of a free amino group with the peroxy radical. The theoretical reaction pathways were speculated to produce five, six, or seven membered ringed structures. The article did not propose an activity for these hypothetical structures nor did it actually identify such a proposed structure in vivo.

PCT/US95/05880, filed by Emory University, discloses that polyunsaturated fatty acids (“PUFAs”) and their hydroperoxides (“ox-PUFAs”) induce the expression of endothelial cell surface adhesion molecule VCAM-1, but not intracellular adhesion molecule (ICAM-1) or E-selectin in human aortic endothelial cells, through a mechanism that is not mediated by cytokines or other noncytokine signals. Specifically, it was disclosed that linoleic acid, arachidonic acid, linoleyl 194 hydroperoxide, and arachidonic hydroperoxide induce cell-surface gene expression of VCAM-1 but not ICAM-1 or E-selectin. Saturated fatty acids (such as stearic acid) and monounsaturated fatty acids (such as oleic acid) do not induce the expression of VCAM-1, ICAM-1 or E-selectin. It was also reported that the induction of VCAM-1 by PUFAs and their fatty acid hydroperoxides is suppressed by dithiocarbamates, including pyrrolidine dithiocarbamate (PDTC), supporting a conclusion that the induction is mediated by an oxidized signal molecule, and that the induction is prevented when oxidation of the molecule is blocked, reversed, or when the redox modified signal is otherwise prevented from interacting with its regulatory target.

PCT/US93/10496, also filed by Emory University, discloses that dithiocarboxylates, and in particular, dithiocarbamates, block the induced expression of the endothelial cell surface adhesion molecule VCAM-1, and are therefore useful in the treatment of cardiovascular disease, including atherosclerosis, post-angioplasty restenosis, coronary artery diseases, and angina, as well as noncardiovascular inflammatory diseases that are mediated by VCAM-1.

It is an object of the present invention to provide a method and kit-for the assessment of the state of lipid peroxidation of a host, as a means to evaluate the host's risk of oxidation-induced disease.

It is also an object of the present invention to provide a commercially viable method and kit for the assessment of the state of lipid peroxidation of a host.

It is another object of the present invention to provide a method and kit for the assessment of the state of lipid peroxidation of a host, as a means to evaluate the therapeutic efficacy of medical treatment for oxidation-induced disease. It is still another object of the present invention to provide a kit for the diagnosis and quantification of the state of lipid peroxidation of a host.

It is yet another object of the invention to provide materials that are useful in the diagnosis and quantification of the state of lipid peroxidation of a host.

It is still another object of the invention to provide a method to evaluate the ability of a drug candidate to lower the state of lipid peroxidation of a host.

It is another object of the present invention to identify and provide new mediators of cellular responses.

It is yet another object of the present invention to provide new therapeutic agents and methods for the mediation of inflammatory responses.

It is another object of the present invention to provide imaging agents and methods for the identification and quantification of inflammatory disorders.

It is still another object of the present invention to provide a method and kit to detect autoantibodies in the plasma of patients with diseases which illicit autoantibodies, including endometriosis.

### SUMMARY OF THE INVENTION

A method and kit for the diagnosis and quantification of the state of oxidation, and more specifically, the level of lipid peroxidation, of a host is provided that includes contacting a host biological sample with an antibody to an antigen formed by the reaction of a lipid hydroperoxide with a primary amine. This method assesses the risk of, or existence of, oxidative damage in the host. The invention also includes ha monoclonal and polyclonal antibodies, as well as antibody fragments, optionally in purified or isolated form, which are useful in this method and kit.

This method is based on the discovery that the modification of proteins and other primary-amine containing compounds by lipid hydroperoxides results in the generation of antigenic epitopes. It has now been discovered that these antibodies are present even in normal plasma, and can be elevated above normal levels in the plasma of patients suffering from oxidation-induced disease states. This test for the level of lipid peroxides in a patient's biological sample is superior to those presently available, in that it assays for an antigen that is present in significant amounts in the host plasma, and can be performed reliably on in vivo samples. In contrast, antigenic aldehyde-modified proteins have not been detected in plasma by antibodies to aldehyde-modified proteins, except in specific cases such as unstable angina. Further, antibodies to the modification of proteins and other primary-amine containing compounds by lipid hydroperoxides are not cross-reactive with aldehyde modified proteins.

As an illustration of the discoveries underlying the present invention, rabbit serum albumin was modified with lipid peroxides to generate a polyclonal antibody. This polyclonal antibody recognizes proteins modified by lipid peroxides, but fails to recognize unmodified proteins. Using immunohistochemistry, it was determined that this antibody recognizes epitopes present in human atherosclerotic lesions, the atherosclerotic arteries of cholesterol fed monkeys, and RAW macrophage cells pre-incubated with lipid peroxide. The antibody was effective in western blot analysis and can be used to detect the presence of modified epitopes even in normal plasma.

The invention includes methods and kits for analyzing the state of lipid peroxidation in a host that includes a suitable amount of an antibody that can be immobilized on a solid support and is preferably labeled with a detectable agent. Antibodies can be immobilized to a variety of solid substrates by known methods. Suitable solid support substrates include materials having a membrane or coating supported by or attached to sticks, beads, cups, flat packs, or other solid support. Other solid substrates include cell culture plates, ELISA plates, tubes, and polymeric membranes. The antibodies can be labeled with a detectable agent such as a fluorochrome, a radioactive label, biotin, or another enzyme, such as horseradish peroxidase, alkaline phosphatase and 2-galactosidase. If the detectable agent is an enzyme, a means for detecting the detectable agent can be supplied with the kit. A preferred means for detecting a detectable agent employs an enzyme as a detectable agent and an enzyme substrate that changes color upon contact with the enzyme. The kit can also contain a means to evaluate the product of the assay, for example, a color chart, or numerical reference chart.

The kit can be designed to be quantitative or qualitative. It can be used in a scientific laboratory, a medical laboratory, or in the field.

It has further been discovered that certain reaction products of lipid hydroperoxides and primary amines exhibit independent biological activity as mediators of cellular responses. The term “oxykine” is used herein to refer to a fluorescent protein or lipid that is generated by the reaction of a lipid hydroperoxide and a primary amine and which can elicit a response from a target cell. Oxykines can be generated extracellularly or can be formed at the cell membrane or even intracellularly to mediate a cellular response. A wide variety of biologically active molecules that have primary amines can be converted to oxykines that have biological activity.

One nonlimiting example of an oxykine is the stable fluorescent product of the reaction between linoleic hydroperoxide (13-HPODE) and an appropriate amino acid group, such as lysine, in albumin or polylysine, or a small molecular weight compound such as phosphatidylethanolamine. These oxykines act as potent inflammatory signals that induce endothelial VCAM-l gene expression through a mechanism that can be suppressed by selective antioxidants. Other cellular responses that may be elicited by oxykines include, but are not limited to, the generation or activation of MCP-1, IL-1, TNF-α, ICAM, MCSF, and E-selectin.

While all primary amines will react with lipid hydroperoxides to form an antigenic species that can be used to generate antibodies to determine the oxidation state of the host, not all peptides or biologically occurring primary amines will form oxykines. This is because what required to elicit an antibody response may differ from what is required to mediate a cellular response.

In another embodiment, a method for assessing oxidative damage in a biological sample is provided that includes the steps of: (i) isolating an antigen formed by the reaction of a lipid hydroperoxide with a primary amine; and then (ii) identifying the primary amine. The nature of the amine may in certain circumstances provide information regarding the location of the oxidative disorder.

### BRIEF DESCRIPTION OF THE FIGURES

FIG. 1 is a bar chart graph of the recognition of rabbit serum albumin and lipid hydroperoxide modified rabbit serum albumin by the antibody of Example 4, as measured in nanograms protein versus optical density at 405 nm.

FIG. 2 is a bar chart graph of the recognition of oxidized-LDL by the antibody of Example 4, as measured in microgram concentration versus optical density units.

FIG. 3 is a bar chart graph of the dose-dependent induction of VCAM-1 (A) and ICAM-1 (B) by oxidized bovine serum albumin expressed as a percent of the maximal TNF-A induced signal.

FIG. 4 is a schematic diagram of a large scale synthesis of 13-HpODE modified oxykine employing a lipid peroxide generating system. The target protein was placed in a 10 kDa molecular weight cut off membrane and the 13-HpODE generating system was changed every 8-16 hours.

FIG. 5 is a Western blot diagram which demonstrates that sLO and linoleic acid are minimal requirements for the formatio of oxSLO.

FIG. 6 is a Western blot diagram that demonstrates detection of immunoreactive 13-HpODE treated samples after 96 hours in a large scale reaction.

FIG. 7 is a graph of the induction of ICAM-1 by 13-HpODE treated sLO as a percentage of TNF induction over time.

FIG. 8 is an HPLC chromatogram which indicates that HpODE modified sLO is more hydrophobic than untreated sLO.

FIG. 9 is a graph of the induction of ICAM-1 by fractions of oxSLO collected by gel filtration chromatography. The graph indicates that fraction ten most strongly induces ICAM-1.

FIG. 10 is a graph of absorbance (280 nm) versus fractions of oxSLO collected by gel filtration chromatography which indicates that biologicallyqactive fractions 9 and 10 comprise only a portion of the total protein resolved by gel filtration chromatography.

FIG. 11 is a bar chart graph of the induction of ICAM-1 by soybean lipoxygenase, linoleic acid, and oxidized soybean lipoxygenase as a percentage of induction of ICAM by TNF.

IG. 12 is a bar chart graph of the induction of ICAM-1 (as a function of percentage induction by TNF) by soybean lipoxygenase and oxidized soybean lipoxygenase synthesized by the large scale method in human aortic endothelial cells (HAEC).

FIG. 13 is a bar chart graph of the induction of ICAM-1 (as a function of percentage induction by TNF) by soybean lipoxygenase and oxidized soybean lipoxygenase which indicates that oxSLO activates cell surface ICAM expression in a dose dependent manner.

FIG. 14 is a bar chart graph of the induction of ICAM-1 (was a function of percentage induction by TNF) by oxidized soybean lipoxygenase. The graph indicates that oxSLO induces ICAM to a greater extent than it induces VCAM.

FIG. 15 is a Northern blot which indicates that oxSLO but not SLO induces the accumulation of VCAM, ICAM, and MCP-1 mRNA in human aortic endothelial cells (HAEC).

FIG. 16 is a Northern blot which indicates the rapid accumulation of VCAM, ICAM, and MCP-1 in human aortic endothelial cells.

### DETAILED DESCRIPTION OF THE INVENTION

I. Method for the Assessment of Oxidative State of Host

A. Generation of Antibody

(i) Lipid Hydroperoxide

A lipid is a water-insoluble, oily or greasy organic substance that is extractable from cells and tissues by nonpolar solvents such as chloroform and ether. The most abundant form of lipid is a triacylglycerol. Fatty acids are characteristic building blocks of lipids. A fatty acid is a long-chain organic carboxylic acid typically having from four to twenty four carbon atoms. Fatty acids do not typically occur free or uncombined in cells or tissue, but instead are bound in a larger molecule, such as a lipoprotein, albumin, triacylglycerol, a wax, phosphoglyceride (for example, a phosphatidylethanolamine, phosphatidylcholine, phosphatidylserine, phosphatidylinositol, or cardiolipin), a sphingolipid (for example, sphingomyelin, cerebrosides, or gangliosides), or sterol and its fatty acid ester. Materials referred to generically as ceroids and lipofuscins include fatty acids.

As used herein the term polyunsaturated fatty acid (a PUFA) refers to a fatty acid (typically C₈ to C₂₄) that has at least two alkenyl bonds, and includes but is not limited to linoleic (C₁₈Δ), linolenic (C₁₈Δ), arachidonic (C₂₀Δ) and eicosatrienoic (C₂₀Δ) acids.

A lipid hydroperoxide, as the term is used herein, refers to:

1. (i) a polyunsaturated fatty acid; or (ii) a molecule that contains a residue of a polyunsaturated fatty acid;

2. in which at least one of the alkenyl bonds has been converted to a hydroperoxide.

Nonlimiting examples of lipid hydroperoxides are:

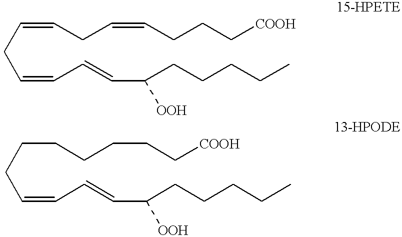

Lipid hydroperoxides can be formed in vivo enzymatically from polyunsaturated fatty acids or lipids containing a PUFA residue by lipoxygenase, cyclooxygenase, peroxidase, or other oxygenase enzyme as well as by non-enzymatic means through the generation of intracellular, extracellular, or cell surface reactive oxygen species. Lipid peroxides can be generated chemically by oxidation of a polyunsaturated fatty acid or lipid containing a PUFA residue using standard methods.

It appears that a wide variety of lipid hydroperoxides can be used to-generate an antibody that reacts with the selected amine to produce an epitope. It appears that this reaction is quite nonselective; virtually any lipid hydroperoxide will react antigenically with a primary amine.

(ii) Primary Amine

Any primary amine can be used to react with the lipid hydroperoxide that forms an antigenic material. It has been discovered that the more hydrophobic and nucleophilic the amine, the more facile the reaction. For example, benzylamine reacts more quickly with a lipid hydroperoxide than ethylamine. On this basis, small amines such as C₁ to C₃ alkyl amines, and ammonia, are not preferred. For administration to a host, a preferred amine is one that exhibits little toxicity either alone or after reaction with the lipid hydroperoxide. The amine can be attached to a larger molecule, for example, a hapten, if desired, to increase an antigenic response.

Synthetic versions of naturally occurring primary amine-containing molecules, such as peptides and proteins, are preferred. Examples include peptides or proteins that have a terminal amino group, a lysine residue (for example, albumin and polylysine) or a histidine residue. Phosphatidylethanolamine, phosphatidylserine, and amine-terminated hormones are also suitable.

That the reaction of the lipid hydroperoxide and proteins occurs naturally in vivo is demonstrated by the fact that commercially available proteins such as bovine serum albumin contain the lipid hydroperoxide/amine epitope. Because of this, it can be preferred to use a synthetic peptide or protein for the preparation of antigen, and ultimately, antibody, for quality control purposes.

(iii) Reaction of Lipid Hydroperoxide with Primary Amine

The ability of a lipid hydroperoxide to react with a protein was demonstrated by Fruebis, Parthasarathy and Steinberg in Proc. Natl. Acad. Sci. USA, Volume 89, pp 10588-10592, November 1992 Medical Sciences. In this article, a lipid hydroperoxide was prepared by reacting linoleic acid or a phospholipid of linoleic acid with soybean lipoxygenase and then the hydroperoxide was incubated with polypeptides in the absence of metal ions. The general approach and experimental conditions set out in the article can be used to prepare a wide variety of lipid hydroperoxide/amine adducts.

The chemical reaction between the lipid hydroperoxide and the primary amine can be carried out in a laboratory or manufacturing plant at room temperature at neutral or near neutral pH, simulating in vivo conditions. Aqueous conditions are preferred, however, the reaction can be run in an organic solvent if the reactants and product are sufficiently organic soluble. The LOOH may be less stable in an organic solvent than in an aqueous solvent. The reaction temperature can be elevated as desired, up to the boiling point of the solvent. The progress of reaction can be monitored by fluorescence spectrophotometry. The excitation of the product is typically between 330 and 360 and emission typically between 420 and 450. The reaction is completed when there is no further increase in fluorescence of the sample. In a typical reaction, the lipid hydroperoxide and the amine are present in a ratio of approximately 1.5:1, however, LQ other ratios can be used as desired to optimize yield or for other purposes.

(iv) Generation and Use of Antibodies

The method and kit for the assessment of the state of lipid peroxidation of a host can include either monoclonal or polyclonal antibodies or antibody fragments.

The term “antibody,” as used herein, includes monoclonal and polyclonal antibodies as well as antibody fragments which bind specifically but reversibly to the described epitope. It is preferred that the antibody or antibody fragment is derived from a monoclonal antibody or antibody fragment. Preparation of monoclonal and polyclonal antibodies to an antigen formed by the reaction of a lipid hydroperoxide with a primary amine can be achieved using any known method, and for example, those described in Zola, H. (1988) “Monoclonal Antibodies—A manual of techniques” CRC Press, and Antibodies: A Laboratory Manual, Harlow & Lane; Cold Spring Harbor (1988), incorporated herein by reference.

In one embodiment, primarily for laboratory use, ale animals are immunized with the antigen, and preferably an adjuvant. Booster immunizations are optionally continued with antigen in PBS and mixed with adjuvant at periodic intervals. The animals are then bled following the immunizations. After removal of clot and debris, the serum can be assayed by ELISA. Monthly, or other periodic titers can be obtained after initial immunization.

Alternatively, spleens are harvested from animals immunized with the antigen, and preferably an adjuvant. Spleen cells are separated and fused with immortal myeloma cells using polyethylene glycol. The fused hybridoma cells are selected and cultured in vitro. The hybridoma cell culture fluids are tested for the presence of hybridoma antibodies having the desired specificity. The selection technique for identifying the appropriate monoclonal or polyclonal antibody is an important aspect in obtaining desired immunospecificity. The hybridoma cells can be tested for the presence of antibodies specific for the antigen, for example, with an ELISA conducted by standard methods.

In general, if the product of the selected lipid a hydroperoxide and the selected amine contains an antigen with one epitope, that antigen will elicit the-production of a monoclonal or a polyclonal antibody. If multiple antigens or an antigen with multiple epitopes are used, a polyclonal antibody will be obtained. For example, when LOOH is reacted with a serum albumin, an antigen with multiple epitopes is produced because albumin has a number of lysine residues at different locations in the molecule. This antigen will elicit the production of polyclonal antibodies. If, in contrast, the reaction product of a simple amine with LOOH is used as the antigen, that antigen may induce the formation of monoclonal or polyclonal antibodies. For example, if a phosphatidylethanolamine is selected that contains an unsaturated lipid moiety, it can an act as both the lipid component and the amine component. Such a molecule may elicit the production of monoclonal or polyclonal antibodies.

It has been observed that proteins from natural sources can contain a small amount of LOOH/amine antigen. Because of this, for quality control purposes, it is preferred to react a synthetic peptide or a synthetic amine-containing molecule with LOOH to form an antigen for antibody production.

The variable heavy (V_{H}) and variable light (V_{L}) domains of the antibody are involved in antigen recognition, a fact first recognized by early protease digestion experiments. Further confirmation was found by “humanization” of rodent antibodies.

Variable domains of rodent origin can be fused to constant domains of human origin such that the resultant antibody retains the antigenic specificity of the rodent parented antibody (Morrison et al. (1984) Proc. Nat. Acad. Sci. USA 81, 6851-6855). “CDR grafting” can be used to humanize rodent antibodies.

Additionally or alternatively, recombinant monoclonal antibodies may be “primatized,” i.e. antibodies formed in which the variable regions are derived from two different primate species, preferably the variable regions of the antibody from the macaque monkey, and the constant regions from human. The advantages of such antibodies include high homology to human immunoglobulin, presence of human effector functions, reduced immunogenicity and longer serum half-life (Newman et al. (1992) Biotechnology 10, 1455).

The region specific for the antigen can be expressed as part of a bacteriophage, for example, using the technique of McCafferty et al. (1990) Nature 348: 552-554. Antibody-like molecules of the invention can be selected from phage display libraries using the methods described in Griffiths et al. (1993) EMBO J. 12, 725-734, in which the antigens are immobilized and used to select phages. Also, appropriate cells grown in monolayers and either fixed with formaldehyde or glutaraldehyde or unfixed can be used to bind phages. Irrelevant phages are washed away and bound phages recovered by disrupting their binding to the-antigen and reamplifying in bacteria. This selection and amplification process is done several times to enrich the phage population for those molecules which are the antibody-like molecules of the invention.

Antibody fragments include Fab-like molecules (Better et al. (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al. (1988) Science 242, 423; Huston et al. (1988) Proc. Natl. Acad. Sci. USA 85, 5879) and single domain antibodies (dAbs) comprising isolated V domains (Ward et al. (1989) Nature 341, 544). A general review of the techniques involved in the synthesis of antibody fragments which retain their specific binding sites is found in Winter & Milstein (1991) Nature 349, 293-299).

Antibody fragments, including but not limited to Fab, (Fab)₂, Fv, scFv and dAb fragments are included in this invention. Antibody-like molecules can be prepared using the recombinant DNA techniques of WO 84/03712.

There can be advantages to using antibody fragments, rather than whole antibodies. Fab, Fv, ScFv and dAb antibody fragments can all be expressed in and secreted from E. coli, thus allowing the facile production of large amounts of the fragments.

Whole antibodies, and F(ab′)₂ fragments are “bivalent.” By “bivalent” it is meant that the antibodies and F(ab′)₂ fragments have two antigen combining sites. In contrast, Fab, Fv, ScFv and dAb fragments are monovalent, having only one antigen combining site.

The art of “antibody engineering” is advancing rapidly, as is described in Tan L. K. and Morrison, S. L. (1988) Adv. Drug Deliv. Rev. 2: 129-142, Williams, G. (1988) Tibtech 6: 36-42 and Neuberger, M. S. et al (1988) 8th International Biotechnology Symposium Part 2, 792-799 (all of which are incorporated herein by reference), and is well suited to preparing antibody-like molecules derived from the antibodies of the invention.

The antibodies can be used for a variety of purposes relating to the study, localization, isolation and purification of the antigen to which they specifically bind. In particular, the antibody can be used in the imaging and treatment of cells exhibiting the antigen. The antibody of the invention can be coupled to a scintigraphic radiolabel, a radioisotope, a cardiovascular, antiinflammatory, or other drug, an enzyme for converting a prodrug into a cardiovascular, an antiinflammatory, or other desired drug, along with that prodrug, or other cellular process mediating compound. Such conjugates have a “binding portion,” which consists of the antibody of the invention, and a “functional portion,” which consists of the radiolabel, drug or enzyme, etc. The binding portion and the functional portion can be separated by a linking moiety.

The binding portion and the functional portion of the conjugate (if also a peptide or polypeptide) can be linked together by any of the conventional ways of cross-linking polypeptides, such as those generally described in O'Sullivan et al. (1979) Anal. Biochem. 100, 100-108. For example, one portion can be enriched with thiol groups and the other portion reacted with a bifunctional agent capable of reacting with those thiol groups, for example the N-hydroxysuccinimide ester of iodoacetic acid (NHIA) or N-succinimidyl-3-(2-pyridyldithio)propionate (SPDP). Amide and thioether bonds, for example achieved with m-maleimidobenzoyl-N-hydroxysuccinimide ester, are generally more stable in vivo than disulphide bonds.

Alternatively, if the binding portion contains carbohydrates, such as would be the case for an antibody or some antibody fragments, the functional portion can be linked via the carbohydrate portion using the linking technology in EP 0 088 695.

The functional portion of the conjugate can be an enzyme for converting a prodrug into a drug, using technology similar to that described by Bagshawe and his colleagues (Bagshawe (1987) Br. J. Cancer 56, 531; Bagshawe et al (1988) Br. J. Cancer 58, 700; WO 88/07378).

It may not be necessary for the whole enzyme to be present in the conjugate but, of course, the catalytic portion must be present. So-called “abzymes” can be used, in which a monoclonal antibody is raised to a compound involved in the reaction one wishes to catalyze, usually the reactive intermediate state. The resulting antibody can then function as an enzyme for the reaction.

The conjugate can be purified by size exclusion or affinity chromatography, and tested for dual biological activities. The antigen immunoreactivity can be measured using an enzyme-linked immunosorbent assay (ELISA) with immobilized antigen and in a live cell radio-immunoassay. An enzyme assay can be used for β-glucosidase using a substrate which changes in absorbance when the glucose residues are hydrolyzed, such as ONPG (o-nitrophenyl-β-D-glucopyranoside), liberating 2-nitrophenol which is measured spectrophotometrically at 405 nm.

Stability of the conjugate can be tested in vitro initially by incubating at 37° C. in serum, followed by size exclusion FPLC analysis. Stability in vivo can be tested in the same way in mice by analyzing the serum at various times after injection of the conjugate. In addition, it is possible to radiolabel the antibody with ¹²⁵I, and the enzyme with ¹³¹I before conjugation, and to determine the biodistribution of the conjugate, free antibody and free enzyme in mice.

Alternatively, the conjugate can be produced as a fusion compound by recombinant DNA techniques whereby a length of DNA comprises respective regions encoding the two portions of the conjugate either adjacent to one another or separated by a region encoding a linker peptide which does not destroy the desired properties of the conjugate. Conceivably, the two functional portions of the compound can overlap wholly or partly. The DNA is then expressed in a suitable host in known ways.

The antibodies or their conjugates can be administered in any suitable way, usually parenterally, for example intravenously or intraperitoneally, in standard sterile, non-pyrogenic formulations of diluents and carriers, for example isotonic saline (when administered intravenously). Once the conjugate has bound to the target cells and been cleared from the bloodstream (if necessary), which typically takes a day or so, if desired, a prodrug can be administered, usually as a single infused dose, or the target area is imaged. If needed, because the conjugate can be immunogenic, cyclosporin or some other immunosuppressant can be administered to provide a longer period for treatment but usually this will not be necessary.

The timing between administrations of conjugate and a prodrug can be optimized in a non-inventive way since diseased tissue/normal tissue ratios of conjugate (at least following intravenous delivery) are highest after several days, whereas at this time the absolute amount of conjugate bound to the target tissue, in terms of percent of injected dose per gram, is lower than at earlier times. Therefore, the optimum interval between administration of the conjugate and a prodrug will be a compromise between peak tissue concentration of antibody/enzyme and the best distribution ratio between affected and normal tissues. The dosage of the conjugate will be chosen by the physician according to the usual criteria. The dosage of any conjugate will be chosen according to normal criteria, particularly with reference to the type, condition and location of the target tissue and the weight of the patient. The duration of treatment will depend in part upon the rapidity and extent of any immune reaction to the conjugate.

The functional portion of the conjugate, when the conjugate is used for diagnosis of inflammation, usually comprises and may consist of a radioactive atom for scintigraphic studies, for example technetium 99m (^{99m}Tc) or iodine-123 (¹²³I) or a spin label for nuclear magnetic resonance (nmr) imaging (also known as magnetic resonance imaging, mri), such as iodine-123 again, iodine-131, indium-111, fluorine-19, carbon-13, nitrogen-15, oxygen-17, gadolinium, manganese or iron.

When used in a conjugate for selective destruction of tissue, the functional portion can comprise a highly radioactive atom, such as iodine-131, rhenium-186, rhenium-188, yttrium-90 or lead-212, which emits enough energy to destroy neighboring cells.

The radio- or other labels can be incorporated in the conjugate in known ways. For example, the label can be synthesized using suitable reactants that contain, for example, fluorine-19 in place of hydrogen. Labels such as ^{99m}TC, ¹²³I, ¹⁸⁶Rh, ¹⁸⁸Rh and ¹¹¹In can be attached via a cysteine residue peptides. Yttrium-90 can be attached via a lysine residue. The IODOGEN method (Fraker et al. (1978) Biochem. Biophys Res. Commun. 80: 49-57) can be used to incorporate iodine-123. “Monoclonal Antibodies in Immunoscintigraphy” (Chatal, CRC Press 1989) describes other methods in detail.

B. Method for Detecting Antigen

The invention includes a method for detecting antigens formed by the reaction of a lipid hydroperoxide with a primary amine. This test can be performed on a laboratory sample or on a biological sample. As used herein the term biological sample refers to a sample of tissue or fluid isolated from a host, typically a human, including, but not limited to, for example, plasma, serum, spinal fluid, lymph fluid, ocular fluid, tissue of the skin, peritoneal fluid, urine, bile, cardiovascular, respiratory, intestinal, genital, uteral, or central nervous system tracts, tears, placenta, umbilical cord, milk, endothelial cells, endometrial cells, epithelial cells, tumors, and organs, and also samples of in vitro cell cultures constituents including but not limited to conditioned medium resulting from the growth of cells in cell culture medium.

According to the present invention, the oxidation state, and in particular, the lipid peroxidation state, of a host, is evaluated by testing a biological sample of the host for the presence and level of either: (I) antigens which bind to an antibody formed as described in Section I. A.; or (ii) antibodies that are crossreactive with the antibodies formed as described in Section I. A. It has been determined that every host, even healthy persons, have both (I) and (ii) in their tissue and fluid in varying amounts. In fact, commercially available bovine serum albumin contains (I) and thus will exhibit a positive response on exposure to antibodies formed according to Section I. A. Therefore, the mere existence of (I) or (ii) in a host is not in itself an indication of an oxidation-induced diseased state. Instead, the level of (I) or (ii) in a host should be considered in light of population and individual norms. Thus the diagnostic is quite similar to that for cholesterol, wherein a host's level is compared to statistical norms. Further, as with cholesterol tests, in certain cases, changes in individual levels can provide more important diagnostic information than the individual level itself.

Typically, if a cholesterol test indicates that the patient may be at risk for heart disease, a second level battery of tests is performed to obtain more diagnostic information, including the absolute serum HDH, LDL, and triacylglyceride levels, At the same cholesterol level, different ratios of each component provide distinct diagnostic and prognostic implications. The present test is similar to this, in that if the level of lipid hydroperoxides indicates that the patient is at risk for or has an inflammatory disorder, including a cardiovascular disorder, it may be appropriate to carry out a series of second level tests to both confirm the diagnosis and to obtain more information regarding the disorder. These second level tests can evaluate the presence and or amount of other surrogate markers of the suspected disease. Surrogate markers include, but are not limited to LDL, HDL, triacylglycerides, L₍ₚ₎A (formed by the reaction of LDL with protein a), VCAM (soluble, circulating VCAM-1 levels are elevated in the serum of patients with systemic inflammatory diseases), ICAM, E-selectin, MCSF, G-CSF, TNF-A, IL-1, and MCP-1. Surrogate markers of other specific diseases are known and assays for these markers are commercially available or available from a medical laboratory.

Alternatively, antibodies specific for target proteins and oxidized surrogate markers can be used to assay specific components of the lipid peroxide modified sample itself, to obtain more information regarding which primary amines are involved in the oxidation process. This provides additional specificity and sensitivity for the diagnosis of atherosclerosis and other inflammatory diseases, and can provide information for the determination of which, if any, lipid peroxidized materials may be acting as oxykines. Using standard double antibody detection techniques (e.g., immunoprecipitation and Western blot), lipid peroxide modified amines, including LDL, HDL, triacylglycerides, L₍ₚ₎A, VCAM, ICAM, E-selectin, MCSF, G-CSF, TNF-α, IL-1, and MCP-1 can be identified and quantitated from tissue or fluid. This component of overall lipid peroxide state may represent a sensitive and specific marker for lipid peroxide mediated vascular inflammatory events characteristic of atherosclerosis. Similarly, lipid peroxide modification of apo-B can be detected using both an apo-B antibody and an LOOH/amine antibody.

Any test which measures the binding of an antigen to an antibody can be used to evaluate the level of antigen or antibody in the host's biological sample according to the present invention. A number of such tests are known and commonly used commercially.

Immunocytochemistry and immunohistochemistry are techniques that use antibodies to identify antigens on the surface of cells in solution, or on tissue sections, respectively. Immunocytochemistry is used to quantify individual cell populations according to surface markers. Immunohistochemistry is used to localize particular cell populations or antigens. These techniques are also used for the identification of autoantibodies, using tissues or cells that contain the presumed autoantigen as substrate. The antibodies are usually identified using enzyme-conjugated antibodies to the original antibody, followed by a chromagen, which deposits an insoluble colored end product on the cell or tissue.

Another common method of evaluation is a radioimmunoassay, in which radiolabelled reagents are used to detect the antigen or antibody. Antibody can be detected using plates sensitized with antigen. The test antibody is applied and detected by the addition of a radiolabelled ligand specific for that antibody. The amount of ligand bound to the plate is proportional to the amount of test antibody. This test can be reversed to test for antigen. Variations of radioimmunoassays are competition RIA, direct binding RIA, capture RIA, sandwich RIA, and immunoradiometric assay (RMA).

Enzyme linked immunoabsorbent assays (ELISA) are a widely used group of techniques for detecting antigen and antibodies. The principles are analogous to those of radioimmunoassays except that an enzyme is conjugated to the detection system rather than a radioactive molecule. Typical enzymes used are peroxidase, alkaline phosphatase and 2-chractosidase. These can be used to generate colored reaction products from colorless substrates. Color density is proportional to the amount of reactant under investigation. These assays are more convenient than RIA, but less sensitive.

The Western blotting (immunoblotting) method is used to characterize unknown antigens. Components of the biological sample are separated -by gel electrophoresis. SDS gels separate according to molecular weight and IEF gels separate the samples according to charge characteristics. The separated proteins are transferred to membranes (blotted) and identified by immunocytochemistry.

Less often used but suitable methods of evaluation include the Farr assay (in which radiolabelled ligands bind to and detect specific antibody in solution which are precipitated and quantified), precipitin reactions (in which antibodies and antigens crosslink into large lattices to form insoluble immune complexes; only works if antigen and antibody are present in sufficient amounts, at near equivalence, and when there are enough epitopes available to form a lattice); nephelometry (measures immune complexes formed in solution by their ability to scatter light); immunodiffusion (detects antigens and antibodies in agar gels); counter-current electrophoresis (similar to immunodiffusion, except that an electric current is used to drive the antibody and antigen together; useful for low concentrations of antigen or antibody); single radial immunodiffusion (SRID)(quantifies antigens by allowing them to diffuse outward from a well into an antibody containing gel; technique can be reversed by diffusing unknown antibody solutions into an antigen-containing well); rocket electrophoresis (similar to SRID, except that the test antigen is moved into the gel by an electric field); and immunofluorescence (similar to immunochemistry, except that it used fluorescence rather than enzyme conjugates). The antibody used to contact the sample of body fluid is preferably immobilized onto a solid substrate. The antibody can be immobilized using a variety of means, as described in Antibodies: A Laboratory Manual, cited supra. Suitable solid substrates include those having a membrane or coating supported by or attached to sticks, synthetic glass, agarose beads, cups, flat packs, or other solid supports. Other solid substrates include cell culture plates, ELISA plates, tubes, and polymeric membranes.

Means for labeling antibodies with detectable agents are also described in Antibodies: A Laboratory Manual, cited supra. The amount of antigen in the host biological sample can be determined by any means associated with the selected assay. For example, the selected immunoassay can be carried out with known increasing amounts of antigen to produce a standard curve or color chart, and then the amount of test antigen can be determined by comparing the result of the test to the standard curve or chart that correlates the amount of antigen-antibody complex with known amounts of antigen. The amount of antigen determined to be present in the host biological sample can be used to evaluate the patient's can be immobilized using a variety of means, as described in Antibodies: A Laboratory Manual, cited supra. Suitable solid substrates include those having a membrane or coating supported by or attached to sticks, synthetic glass, agarose beads, cups, flat packs, or other solid supports. Other solid substrates include cell culture plates, ELISA plates, tubes, and polymeric membranes.

Means for labeling antibodies with detectable agents are also described in Antibodies: A Laboratory Manual, cited supra. The amount of antigen in the host biological sample can be determined by any means associated with the selected assay. For example, the selected immunoassay can be carried out with known increasing amounts of antigen to produce a standard curve or color chart, and then the amount of test antigen can be determined by comparing the result of the test to the standard curve or chart that correlates the amount of antigen-antibody complex with known amounts of antigen. The amount of antigen determined to be present in the host biological sample can be used to evaluate the patient's condition in a number of ways. First, the level of antigen can be compared to a population norm based on statistical data. Second, the level of antigen can be considered in light of the patient's own history of antigen level.

C. Kits

This invention also includes a kit for the diagnosis of the state of lipid peroxidation in a sample. The kit optimally includes an antibody that reacts with an epitope formed by the reaction of a lipid hydroperoxide with an amine found in the biological sample. The antibodies are present in the kit in an amount effective to bind to and detect substantially all of the antigen in the sample. The preferred kit contains sufficient antibody to bind substantially all of the antigen in the sample in about ten minutes or less. The antibody can be immobilized on a solid support, and can be labeled with a detectable agent, as described above. The kit optionally contains a means for detecting the detectable agent. If the antibody is labeled with a fluorochrome or radioactive label, no means for detecting the agent will typically be provided, as the user will be expected to have the appropriate spectrophotometer, scintillation counter, or microscope. If the detectable agent is an enzyme, a means for detecting the detectable agent can be supplied with the kit, and would typically include a substrate for the enzyme in sufficient quantity to detect all of the antigen-antibody complex. One preferred means for detecting a detectable agent is a substrate that is converted by an enzyme into a colored product. A common example is the use of the enzyme horseradish peroxidase with 2,2′-azino-di-[3-ethyl-benzothiazoline sulfonate] (ABTS).

The kit can optionally contain a lysing agent that lyses cells present in the sample of body fluid. Suitable lysing agents include surfactants such as Tween-80, Nonidet P40, and Triton X-100. Preferably, the lysing agent is immobilized onto the solid support along with the antibody.

The kit can also contain a buffer solution for washing the substrate between steps. The buffer solution is typically a physiological solution such as a phosphate buffer, physiological saline, citrate buffer, or Tris buffer.

The kit can optionally include different concentrations of a preformed antigen to calibrate the assay. The kit can additionally contain a visual or numeric representation of amounts of antigen in a calibrated standard assay for reference purposes. For example, if an assay is used that produces a colored product, a sheet can be included that provides a depiction of increasing intensities associated with differing amounts of antigen.

Alternatively, the kit can include an antigen formed by the reaction of a lipid hydroperoxide with an amine for the evaluation of the level of antibody in the biological sample.

The kit can optionally include two antibodies in the detection system. The first antibody which is present in small amounts is specific for the antigen being assayed for. The second antibody provided in higher amounts is used to detect the first antibody. For example, a rabbit antibody can be used to detect the LOOH/amine antigen, and then an anti-rabbit IgG antibody can be used to detect the bound rabbit antibody. Goat antibodies and anti-antibodies are also commonly used.

As one nonlimiting example, a kit for the detection of the lipid peroxidation state of a patient is provided that includes a rabbit antibody specific for the LOOH/amine antigen, anti-rabbit IgG antibody in sufficient amounts to detect the bound first antibody, an enzyme conjugated to the second antibody and a substrate for the enzyme which changes color on exposure to the enzyme.

D. Uses for Diagnostic Kits

The diagnostic method and kits described herein can be used to evaluate a wide variety of medical conditions which are mediated by oxidation-induced events, and in particular, by lipid hydroperoxides. For example, the presence and elevated concentration of lipid hydroperoxides, or reaction products of a lipid hydroperoxide with a primary amine, can be used as an initial diagnostic marker for cardiovascular disease, including atherosclerosis, inflammatory disease, endometriosis, glymerol nephritis, preeclampsia, central nervous system disorders mediated by lipid peroxidation, Alzheimer's disease, psoriasis, asthma, atopic dermatitis, solid tumors, Kaposi's sarcoma, neurodegenerative disease, inflammatory bowel disease (Crohn's disease), rheumatoid arthritis, and ischemia reperfusion. The biological sample can be selected based on the disease to be treated. For example, a tissue specimen in the afflicted area may be optimum if the disease is tissue specific. If this method and kit indicates an increased or abnormal level of lipid peroxidation in the host, additional tests can be employed to confirm the specific disorder.

E. Examples of Assessment of Oxidative State of Host

Rabbit serum albumin (RSA), soybean lipoxygenase, linoleic acid, goat anti-rabbit IgG conjugated with alkaline phosphatase, octylglucoside, tetramethoxypropane and p-nitrophenyl phosphate were obtained from Sigma Chemical Company (St. Louis, Mo.). Nonenol was purchased from Aldrich Chemical Company (Milwaukee, Wis.). Non-fat milk powder was purchased from Bio-Rad Chemical Company (Hercules, Calif.). Tween-20 and 96 well microtiter plates were purchased from Fisher Chemical CA Company (Pittsburgh, Pa.). A 20 amino acid peptide of the sequence, YVTKSYNETKIKFDKYKAKSHEDEL (wherein K stands for lysine) (SEQ I.D. No. 1) was obtained.

### EXAMPLE 1

### Lipid Peroxide Modified Protein Preparation

Linoleic acid was converted into 13-hydroperoxy linoleate by treatment with soybean lipoxygenase (SLO) as described by Fruebis, Parthasarathy, and Steinberg, supra. Linoleic acid hydroperoxide was immediately reacted with immunoglobulin-free RSA and incubated at 37° C. for 2 days. In a typical reaction, 100 nmol of linoleic acid was treated with 30 units of SLO in 1 ml of phosphate buffered saline (PBS) and the reaction was followed by measuring the increase in absorption at 234 nm. Usually, the reaction is complete within 30 minutes. The lipid hydroperoxide (13-HPODE) was then treated with 100 μg of lipid, IgG, and other protein free albumin in the presence of 50 μM EDTA for 2 days at 37° C. The formation of fluorescent products was established by measuring the fluorescence under excitation wavelength of 330 nm and the emission at 430 nm. The product was extracted with chloroform and methanol by the method of Bligh and Dyer (Bligh, et al., Can. J. Biochem. Physiol. 37:911-917 (1959)) to remove unreacted LOOH and then washed several times with ice cold acetone. The final product, LOOH modified RSA(LOOH/RSA) was soluble in aqueous solution and has fluorescent characteristics similar to that of oxidized low-density lipoproteins (Ox-LDL). The generation of LOOH as well as the modification of the protein was performed in the absence of any added metals to limit the formation of aldehydes.

### EXAMPLE 2

### Preparation of Oxidized LDL

LDL was isolated from heparinized plasma of normal human donors using a table-top Beckman TL-100 ultracentrifuge and a TLA-100.4 rotor (Santanam, et al., J. Clin. Invest 95:2594-2600; 1995). A single-spin gradient was used to purify LDL from albumin contamination. The isolation was complete within three hours of obtaining the plasma. The isolated LDL was dialyzed against phosphate-buffered saline (PBS) at 4° C. (200×volumes) for 6 hours. The purity of the isolated LDL was confirmed by the presence of a single band on agarose gel electrophoresis and of intact apolipoprotein B on sodium dodecylsulfate polyacrylamide gel electrophoresis. Isolated LDL (100 μg/ml) was incubated in phosphate-buffered saline with 5 μM copper and incubated in 50 mm plate at 37° C. After 24 hours of incubation the solution was transferred to a glass tube and delipidated by the extraction of the lipids by the method of Bligh and Dyer. The delipidated LDL protein was dissolved in octylglucoside (100 μl of 10 mg/ml was added to the protein, along with 5 μl of 1 N NaOH) as described by Parthasarathy et al. (Parthasarathy, et al., Proc. Natl. Acad. Sci. USA 84:537-540; 1987).

### EXAMPLE 3

### Preparation of Aldehyde Modified Proteins

Globulin free RSA containing 1 mg protein (or other proteins) in 100 μl of PBS was prepared, and the total volume was then increased to 1 ml with PBS. Nonenol or hexanol (100 μl of 25mμM in ethanol), was added to the protein, mixed well, and incubated at 37° C. for 24 hours. Four ml of ice cold acetone was added to the solution and the tube was kept in the freezer for one hour. After centrifugation at 1500 rpm for 10 minutes at 4° C. the supernatant was removed. This step was repeated three more times, and then the precipitate was dried in the vacuum. One ml of PBS was added to the tube, and after mixing the solution was used in an enzyme linked immunoabsorbent assay (ELISA).

Malondialdehyde-modified proteins were prepared as follows. One 100 μl sample of tetramethoxypropane was prepared and 0.5 ml of 6 N HCl was added to the tube. The tube was heated at 60° C. for 30 minutes. The pH was adjusted to 6.4 using 4 N NaOH, and the total volume adjusted to 2.7 ml using PBS. Then 25 μl of the prepared solution was added to 2 mg of free globulin rabbit serum albumin or other proteins, and incubated at 37° C. After 3 hours of incubation the solution was dialyzed against PBS and used in ELISA.

### EXAMPLE 4

### Preparation of Antibody

Three male rabbits with 3-4 kg body weight were purchased from Myrtle Rabbitry (Thompson Station, Tenn.). For primary immunization, 1.5 mg/ml of lipid peroxide modified rabbit serum albumin were dissolved in PBS, mixed with Freunds complete adjutant (Sigma, St. Louis, Mo.), and then injected subcutaneously. Booster immunization was continued with antigen in PBS and mixed with Freunds incomplete adjuvant at 4 week intervals. Rabbits were bled 10 to 14 days following the immunizations and blood was allowed to stand for 4 hours at room temperature and 4° C. overnight. After removal of clot and debris by centrifuging 20 minutes at 3000 rpm, the serum was assayed by ELISA and stored at -200C. Monthly titers were followed and blood was drawn terminally at 6 months after initial immunization. The LOOH/RSA antibody titer in the animals increased with time and plateaued at about four months.

### EXAMPLE 5

### ELISA Assay for LOOH-Modified Proteins

Using ELISA, it was investigated whether the LOOH/RSA antibody recognized the LOOH/modified protein and unmodified protein. Unmodified IgG-free RSA was used as control. incubation at 37° C., wells were washed with PBS three times. Goat anti-rabbit IgG conjugated with alkaline phosphatase was diluted to 1:38,000 and added. After 2 hrs incubation at 370C, wells were washed again and p-nitrophenolphosphate was added. Color development was determined by a plate reader (Anthos II).

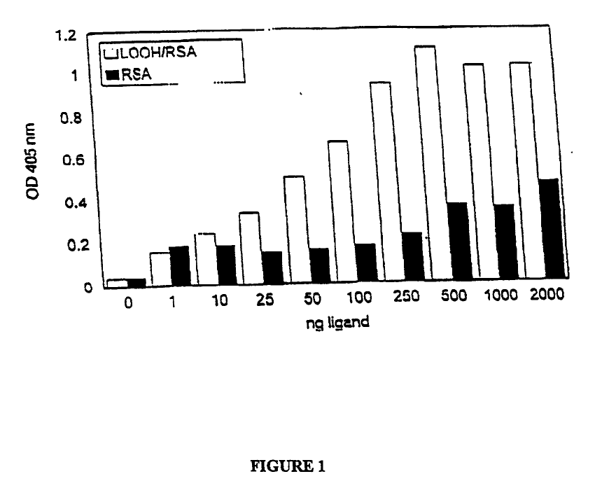

FIG. 1 is a bar chart graph of the recognition of rabbit serum albumin and lipid hydroperoxide modified rabbit serum albumin, as measured in nanograms protein versus optical a density at 405 nm. As illustrated, the antibody at 1:250 dilution selectively recognized the modified protein in a concentration-dependent manner. As little as 10 ng of RSA (less that 2 nM modified RSA) was recognized at significantly higher levels than that of the control protein. Unmodified control protein was barely recognized by the antibody even at high concentrations Controls without the primary or the secondary antibodies were not recognized by the antibody. Other modified proteins (LOOH-modified bovine and human serum albumin, catalase and cytochrome C) were also recognized by the antibody. The apoprotein B₁₀₀ (apo B) component of LDL undergoes extensive modification during the oxidation of LDL. A number of studies have demonstrated that the modification includes new antigenic epitopes generated by the covalent modification of lysine residues by aldehydes.

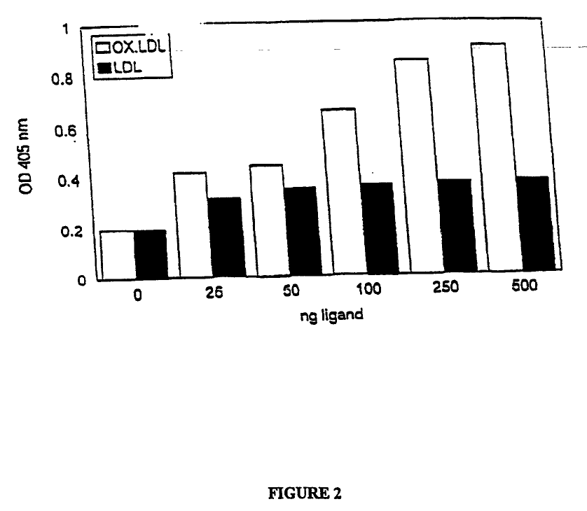

In order to determine whether Ox-LDL also contains lysines modified by intact LOOH, it was investigated whether the antibody to LOOH/RSA recognizes LDL or Ox-LDL. FIG. 2 is a bar chart graph that illustrates the recognition of ox-LDL by the antibody of Example 4 as measured in concentration in micrograms versus optical density units. As indicated in FIG. 2, Ox-LDL was recognized by the antibody in a concentration-dependent manner. The antibody was able to recognize as little as 0.25 μg of Ox-LDL protein (>0.5 nM apo B). Native LDL, prepared in the presence of butylated hydroxytoluene (BHT) was not recognized even at 2.5 μg concentrations. In separate experiments it was determined that the lipid component of Ox-LDL was also recognized by the antibody, suggesting that the amino phospholipids of Ox-LDL, such as phosphatidylethanolamine may be similarly modified.

### EXAMPLE 6

### Immunohistochemistry

The ability of the antibody to recognize modified proteins in tissues was tested under two conditions. In the first study RAW cells, preincubated with LOOH, were used. In the second study, atherosclerotic arteries from cholesterol-fed monkeys were immunostained with the antibody.

Raw macrophages were incubated with 100 μM 13-HPODE for 1, 2 or 3 days as follows. Confluent cells in 6 well plates were treated with 13-HPODE for 1, 2 or 3 days in serum-free DMEM (Dulbecco's modified Eagle's minimum essential medium). Fresh 13-HPODE was added on the second and third day to respective cell dishes. At the end of the first, second or third day cells were fixed with Bouins solution for 10 minutes and immunocytochemistry was performed using antibodies against LOOH/RSA. After fixation, the cells were washed 3 times with PBS. Anti-LOOH/RSA antibody was diluted to 1:250 with PBS containing 3% BSA was added to each well. For negative control, primary antibody was not added. After two hour incubation at room temperature, wells were washed with PBS three times and goat anti-rabbit IgG conjugated with alkaline phosphatase was diluted to 1:100 and added. After 2 hour incubation at room temperature, wells were washed again and incubated with fast red. After color development, the reaction was terminated and the cells were photographed using a Nikon microscope with a camera attachment.

Frozen segments of the abdominal aorta from a group of male cynomolgus monkeys were evaluated by immunohistochemistry. The animals had been fed a moderately high fat diet for over five years consisting of high protein monkey chow supplemented with 8.2% dried egg yolk and 10% lard. The final diet contained 37.5% saturated fat, 44.9% monounsaturated fat, 17.5% polyunsaturated fat with 0.25% cholesterol. The average serum level of these animals was 306 mg/dL. These cholesterol levels are sufficient to produce a range of atherosclerotic lesions in the monkeys. Human aortas displaying different degrees of atherosclerosis development were obtained from organ donors at the time of tissue harvest and were collected with the approval of the Emory University Human Subjects Committee. Human and cynomologus monkey aortas were fixed in paraformaldehyde and frozen in O.C.T. prior to sectioning to 5 μm on a cryostat. Tissue sections were then immunostained using the antibody at a dilution of 1:500 followed by biotinylated goat anti-rabbit IgG (Fisher Scientific) used at a dilution of 1:200 and visualized by the Vector ABC-AP system using Vector Red as a chromagen (Vector Laboratories). Immunohistochemistry demonstrated that cells incubated with LOOH were immunostained with the antibody and the antigenic epitopes were present intracellularly. Control cells and controls lacking in the primary antibodies failed to show immunoreactivity. These arteries showed intense immunoreactivity. The immuno reactivity was localized in areas rich in foam cell macrophages as determined by counter staining for macrophages.

### EXAMPLE 7

### Western Blot Analysis

Western blot analysis was performed after separation of proteins in a 7.5% crosslinked acrylamide gels using 2.5 μg protein. Transblotted samples were detected using 1:250 diluted anti-LOOH/RSA as the primary antibody and a peroxidase-conjugated goat anti-rabbit IgG as the secondary antibody. The cross reactive bands were visualized by chemilumenescence detection.

Using Western blot analysis of LDL and Ox-LDL, it was confirmed that the antibody of Example 4 was able to recognize Ox-LDL but not native LDL. Western blot analysis of normal human plasma showed that at least three different proteins are recognized by the antibody. The plasma samples were prepared in the presence of BHT and it is unlikely that these epitopes were generated in vitro. The identity of these proteins are unknown, although from the mobility on the gel it is suspected that these proteins may represent albumin and apo B products.

### EXAMPLE 8

### Cross-Reactivity of Antibody of Example 7 with Aldehyde-Modified Proteins

A number of antibodies have been described for aldehyde-modified proteins. Since the incubation of the LOOH with the protein is of long duration, it is possible that aldehydes could have contributed to the generation of the antigenic epitopes. In order to establish whether the antibody to LOOH/RSA recognizes proteins modified by aldehydes, a twenty amino acid peptide of the sequence, YVTKSYNETKIKFDKYAEKSHEDEL (wherein K stands for lysine) (SEQ I.D. No.: 1) was prepared. This peptide was used because plasma proteins, such as albumin, from commercial sources may already possess similar epitopes, either as a result of in vivo generation or as a result of generation during in vitro purification procedures. Data from the ELISA of MDA, hexanal, nonenal, and LOOH-modified synthetic peptide is provided in Table 1. The antibody failed to recognize the synthetic peptide or its aldehyde-modified derivatives to any significant extent. In contrast, the LOOH-modified synthetic peptide was avidly recognized by the antibody.

TABLE 1
Recognition of LOOH-modified peptide and not aldehyde-
modified peptides by anti-LOOH/RSA antibody.

| | Native | LOOH- | | Nonenal- | Hexanal- |
|---|---|---|---|---|---|
| μg Peptide | peptide | Peptide | MDA-Peptide | Peptide | Peptide |
| 0 | 57 | 51 | 53 | 51 | 55 |
| 0.25 | 46 | 190 | 50 | 56 | 64 |
| 1.25 | 38 | 327 | 57 | 60 | 84 |
| 2.5 | 42 | 358 | 62 | 46 | 103 |

Microtiter wells were coated with increasing concentrations of unmodified, or modified peptide. After blocking wells with non-fat milk powder, 100 μl of 1:250 dilution of anti-LOOH/RSA antibody was added to each well. After wash, anti-rabbit IgG conjugated with alkaline phosphatase was added to each well. After adding substrate p-nitrophenyl phosphate, the OD was measured at 405 nm using a microplate reader. Values represent averages of a triplicate set of optical density readings of wells from one of at least 2 separate trials. 20 Western blot analysis of the aldehyde- and LOOH-modified peptide confirmed that none of the aldehyde-modified synthetic proteins were recognized to any significant extent, whereas LOOH-modified peptide was recognized by the antibody.

II. Biological Activity of Oxykines

A. Definition of Oxykines

It has been discovered that certain reaction products of lipid hydroperoxides and primary amines exhibit independent biological activity as mediators of cellular responses. The term “oxykine” is used herein to refer to a fluorescent protein or lipid that is generated by the reaction of a lipid hydroperoxide and a primary amine and which can elicit a response from a target cell. Oxykines can be generated extracellularly or can be formed at the cell membrane to mediate a cellular response. Oxykines can also be generated intracellularly.

A wide variety of biologically active molecules that have primary amines can be converted to oxykines that have biological activity. One example of an oxykine is the stable fluorescent product of the reaction between linoleic hydroperoxide (13-HPODE) and an appropriate amino acid group, such as lysine, in albumin or polylysine, or a small molecular weight compound such as phosphatidylethanolamine. Certain oxykines act as potent inflammatory signals that induce endothelial VCAM-1 gene expression through a mechanism that can be suppressed by selective antioxidants. Other cellular responses that may be elicited by oxykines are the generation or activation of MCP-1, IL-1, TNF-α, ICAM, MCSF, and E-selectin.

Oxykines can be used as mediators of cellular inflammatory responses, including cardiovascular responses since the oxidation of lipids is associated with inflammatory and cardiovascular disease. Specific disease states that are inflammatory or cardiovascular in nature include atherosclerosis, endometriosis, glymeral nephritis, preeclampsia, central nervous system disorders mediated by lipid peroxidation, Alzheimer's disease, autoimmune disorders, psoriasis, asthma, atopic dermatitis, skin cancer, neurodegenerative disease, irritable bowel disease (Crohn's disease), rheumatoid arthritis, ischemic reperfusion, osteoarthritis, asthma, dermatitis, multiple sclerosis, angioplasty restenosis, coronary artery diseases, and angina.

While all primary amines will react with lipid hydroperoxides to form an antigenic species that can be used to generate antibodies to determine the oxidation state of the host, not all peptides or biologically occurring primary amines will form oxykines. This is because only one epitope is required to elicit an antibody response, however, to mediate a cellular response, the number and location of reacted sites is critical for complementary binding to a receptor.

Nonlimiting examples of oxykine epitopes are disclosed in Proc. Natl. Acad. Sci. USA, Volume 89, pp 10588-10592, November 1992 Medical Sciences, incorporated herein by reference. In particular, the epitopes can include:

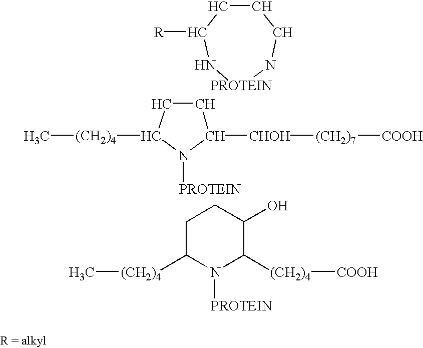

R=alkyl

The antibodies of the present invention can be used to block the damaging activity of antigen, particularly by physically interfering with its ability to mediate a cellular response.

B. In Vitro Synthesis and Characterization of the Oxykine

Lipid modification of soybean lipoxygenase (sLO) was achieved by incubation of lipid hydroperoxide with lipoxygenase using a modified method of Freubis, Parthasarathy, and Steinberg (Proc. Natl. Acad. Sci. USA, 89, 10588-10592). For this L(synthesis, maintaining a high lipid to protein ratio was important. In a small scale reaction (1 ml), 250 μM linoleic acid was incubated directly with sLO (50 units, 80 ng) at room temperature for three days; agitation of the sample was important for the introduction of air into the reaction. All reactions were-run in 10 mM Tris, pH 8.0, 15 mM sodium chloride. The unpurified reaction mix was used to generate rabbit polyclonal antibodies and mouse monoclonal antibodies against the oxidatively modified soybean lipoxygenase oxykine (oxSLO).

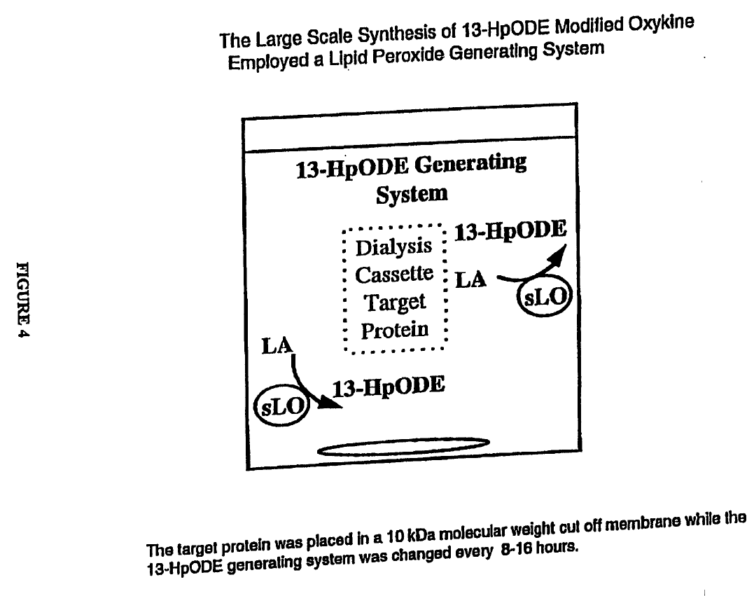

In scaling up the reaction, it became impossible to maintain the large lipid to protein ratio due to problems of immiscibility. A method was developed for the synthesis of the 13-HpODE modified lipoxygenase oxykine, or modification of the “target” protein (see FIG. 4). The target protein, 1-3 mg in a volume up to 1.5 ml, was sequestered within a 10 kDa molecular weight cut off membrane. This allowed for the continued exchange of the 250 ml lipid peroxide generating system with retention of the protein target. Catalytic amounts of soybean lipoxygenase, 1,260 U, and 800 μM linoleic acid were used in the peroxide generating system to catalyze the formation of 13-hydroperoxy-[S-(E,Z)]-9,11-octadecadienoic acid. The generating system was exchanged with a fresh sLO/linoleic acid every eight to sixteen hours for a period of at least 170 hours. The reaction was performed in 10 mM Tris, pH 8.0, 15 mM sodium chloride with constant stirring. One advantage of the large scale method was that any protein larger than 10 kDa or a mixture of proteins can be used as the target protein.

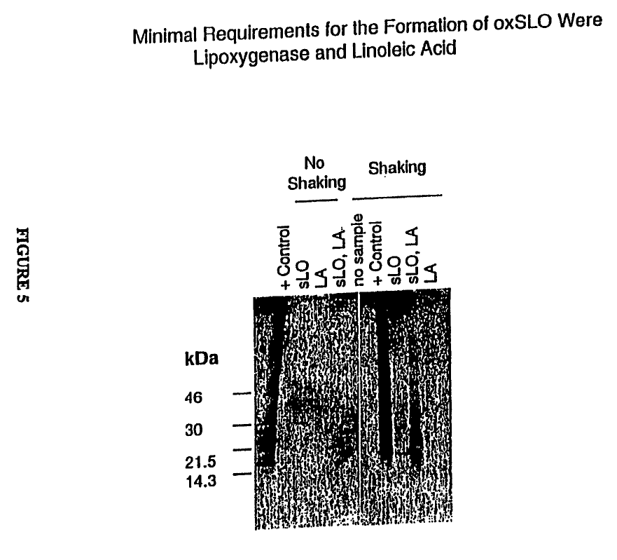

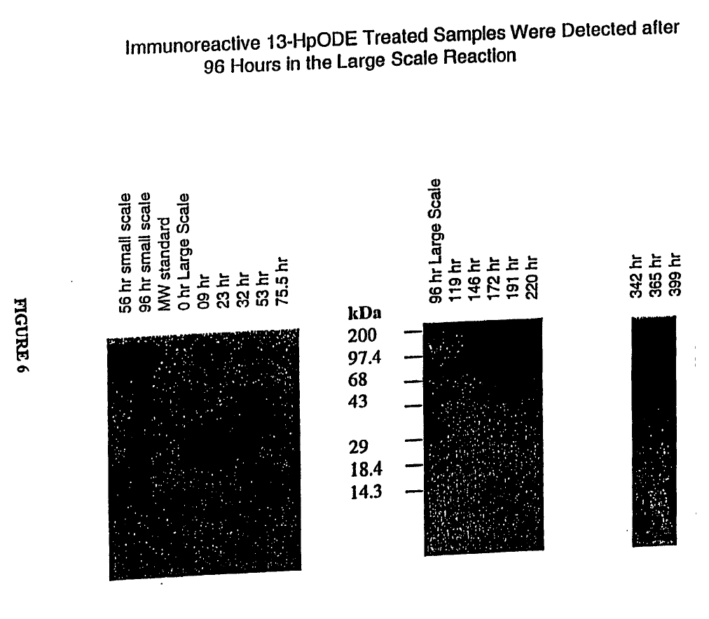

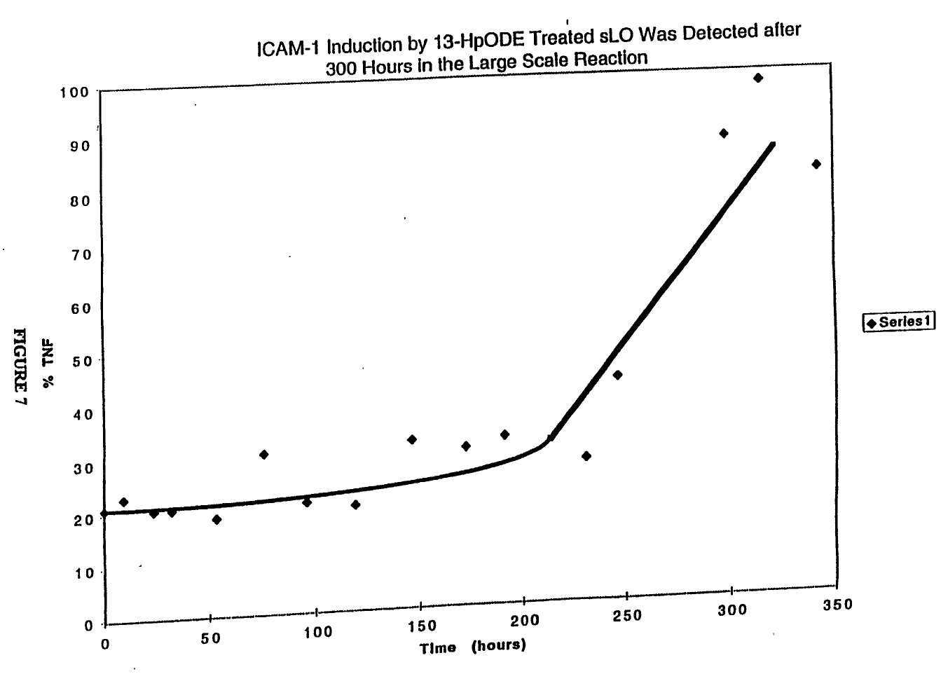

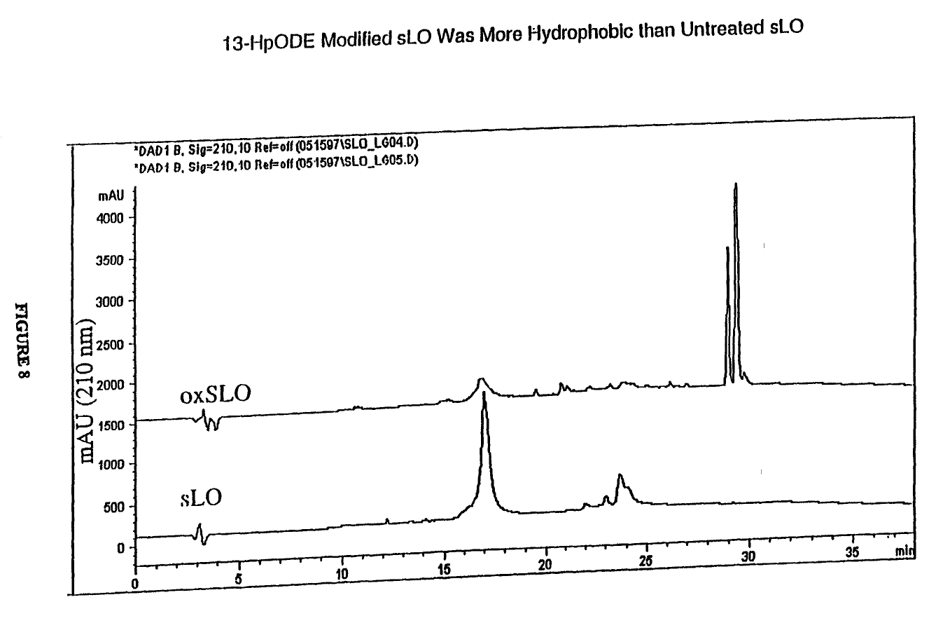

Criteria for the formation of 13-HpODE oxidized oxykines include induction of ICAM-1 in the endothelial cell based assay (to be discussed in detail in the later sections of this report) and immunoreactivity with oxykine antibodies. The minimal requirements for the formation of the oxykine were assessed using the small scale reaction. As observed by Western analysis using the polyclonal antibody, sLO and linoleic acid were minimal requirements for the three day reaction (see FIG. 5). Shaking the reaction enhanced the yield. There was no cross reactivity when sLO was incubated with buffer alone. The time course for the formation of oxykine in the large scale reaction was followed by immunoreactivity and biological activity (induction of ICAM-1; see FIGS. 6 and 7). Immunoreactivity activity was observed prior to biological activity. The initial product observed by the Western analysis was a high molecular weight band migrating at approximately 80 kDa. The reaction products observed at later time points formed a ladder with a predominant species at approximately 25 kDa. The time course of the reaction by both immunoreactivity and biological activity was dependent on the concentration of lipid; increased linoleic acid concentration increased the rate of reaction.

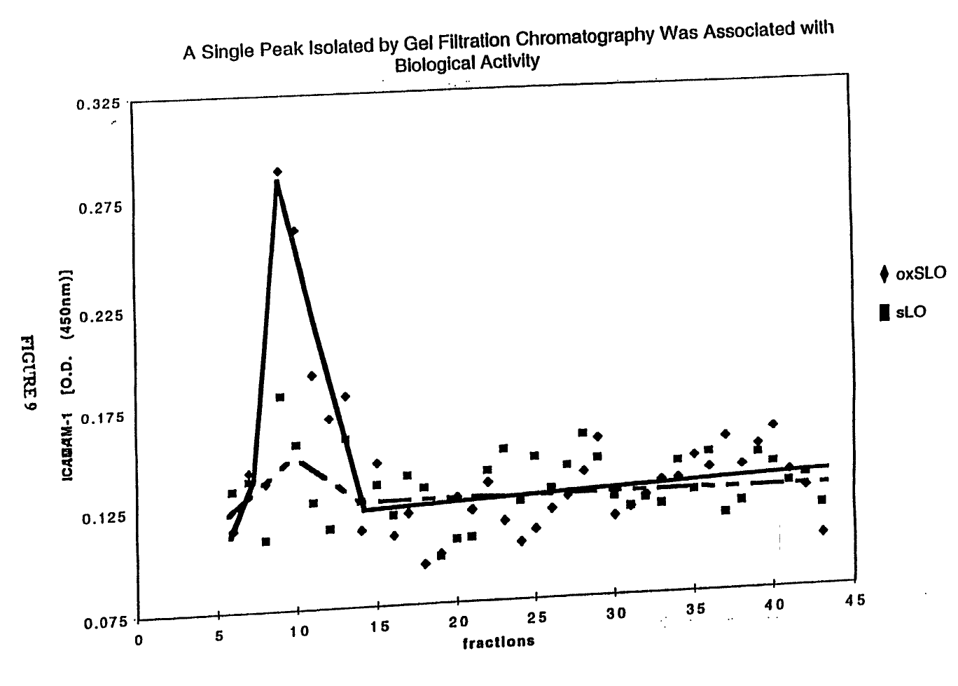

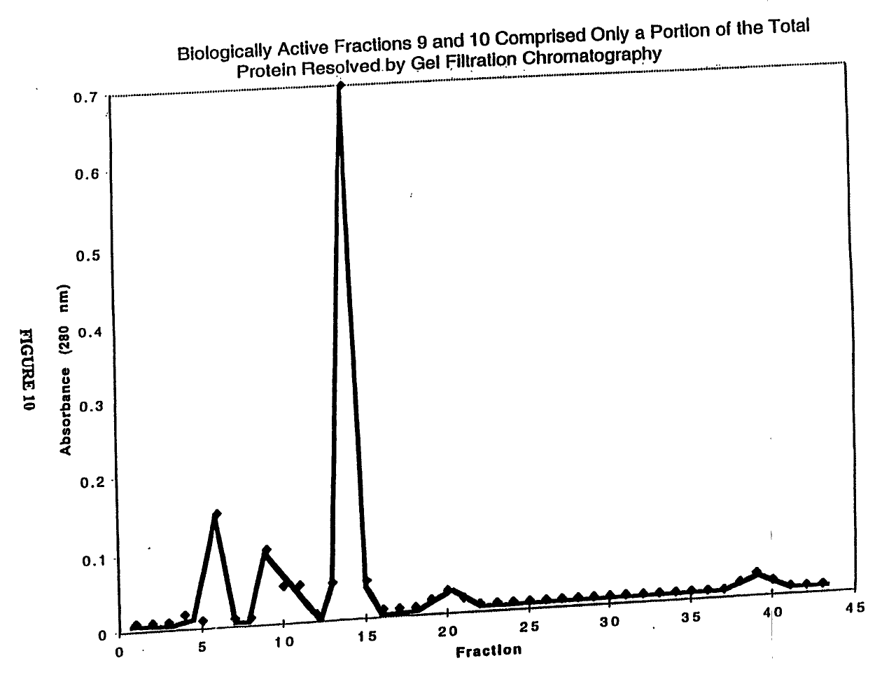

Oxykine reaction products were examined by analytical reverse phase high performance liquid chromatography (RP-HPLC) and gel filtration. HPLC analysis using a C18 column revealed that the reaction products were more hydrophobic than the starting materials, consistent with the addition of the lipid to the lipoxygenase (see FIG. 8). Preparative gel filtration was used to partially purify the oxykine (see FIGS. 9 and 10). Biological activity was found in fractions 9 and 10 although the majority of the protein was found in other fractions.

Stability of the oxykine was assessed on the basis of immunoreactivity and biological activity. The oxykine was stable to a number of acidic and basic conditions and to the presence of reducing agents, as observed by Western analysis. Upon pre-incubation with 3.5 M MgCl₂, 10 mM sodium phosphate, pH 7.2; 5.0 M LiCl, 10 mM sodium phosphate, pH 7.2; 100 mM triethylamine, pH 11.5; and 100 mM glycine, pH 2.5, immunoreactivity with the polyclonal and monoclonal antibodies was retained. In contrast, incubation with 3.5 M MgCl₂, 10 mM sodium phosphate, pH 7.2 caused a loss of biological activity while the remaining storage conditions had no effect. Whether the loss of activity was due to a change in-protein conformation, oxidation state, or other cause remains to be determined. Biological activity was maintained after heat denaturation and heat denaturation in the presence of dithiothreitol. Addition of EDTA, Trolox, and probucol also had no effect on ICAM-1 induction. Following protein acid hydrolysis, biological activity was lost.

### EXAMPLE 9

### Characterization of the Biological Activity of the oxSLO: oxSLO Induced ICAM Expression in Human Aortic Endothelial Cells (HAEC)

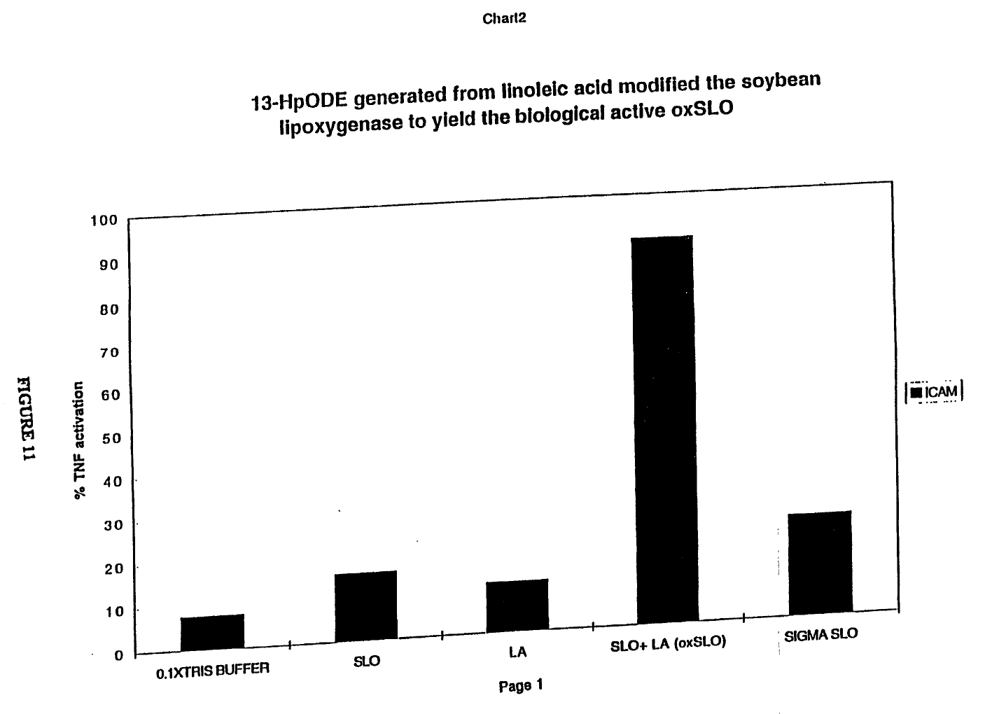

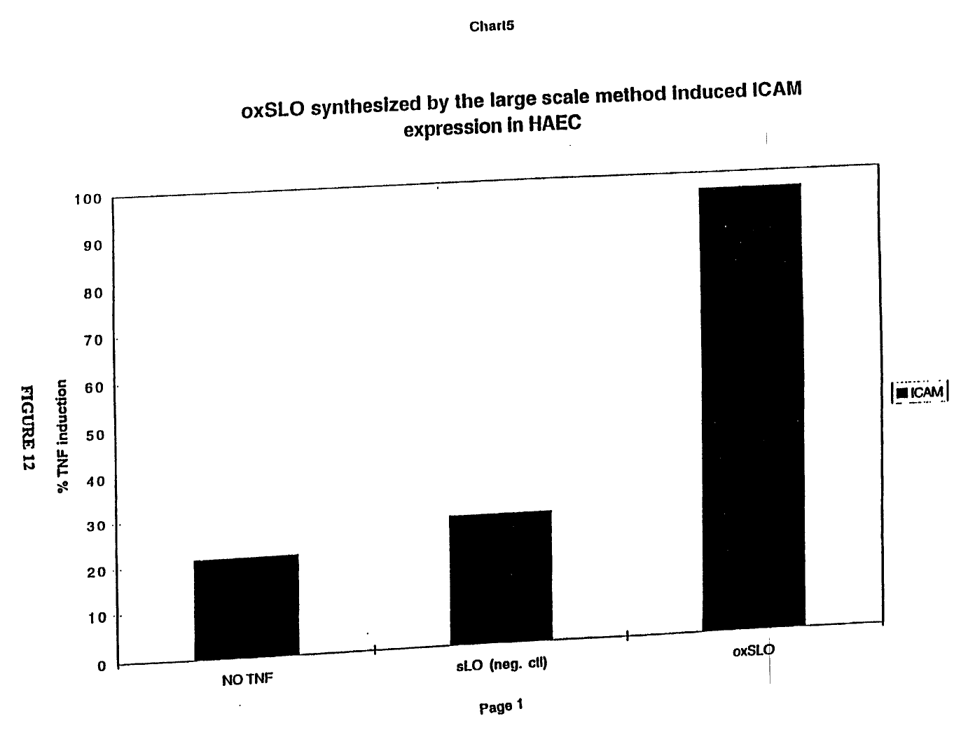

In a small scale synthesis reaction, sLO converted 250 uM linoleic acid (LA) into 13-HpODE within the first hour of room temperature incubation. During the subsequent 3 days incubation, 13-HpODE presumably oxidatively modified the enzyme and generating epitopes on the enzyme which can elicit biological s activity such as the expression of cell surface ICAM on HAEC as shown in ELISA assay depicted in FIG. 11. HAEC grown on microtiter plates were exposed to treatment for 16 hr. before the ELISA assay. sLO or LA alone after 3 days of incubation did not become active suggesting the oxidative modification of sLO is essential to the creation of the biological active epitope. In the large scale synthesis reaction, only in the presence of the 13-HpODE generating system did the sLO become active as shown in FIG. 12. sLO negative control represented the sLO enzyme incubated with buffer alone for the extended period of time. We have confirmed the oxSLO synthesized by either method are functionally and immunologically identical. Furthermore, we have determined that formation of a biologically activity is protein specific. Reaction of rabbit serum albumin using the large or small scale oxykine reaction generates immunoreactive but not biologically active products.

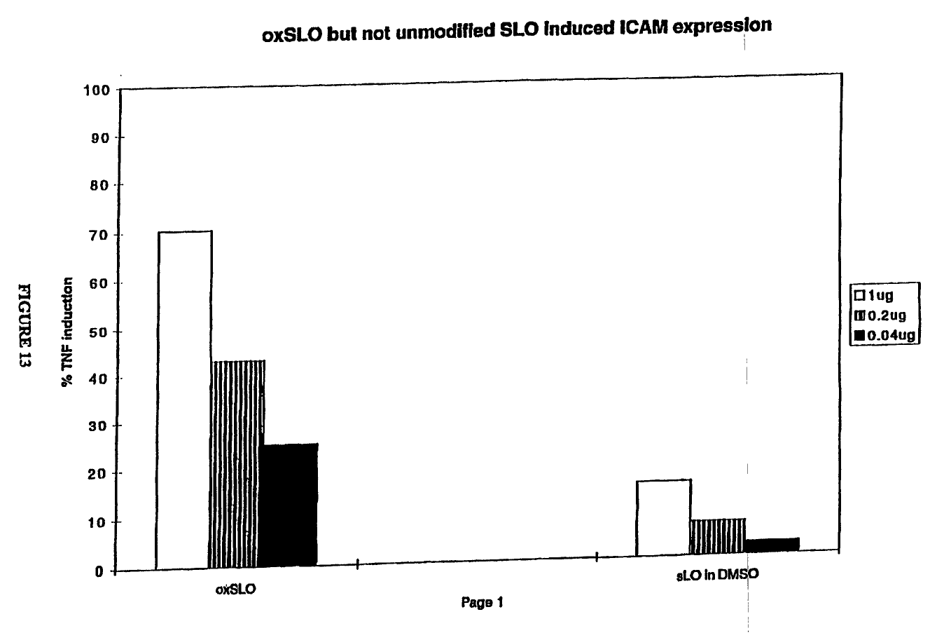

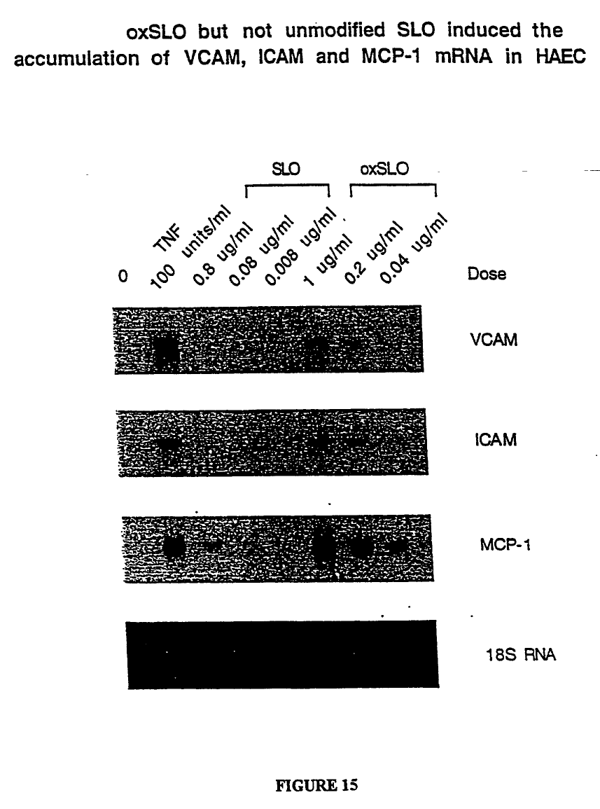

The oxSLO activated cell surface ICAM expression was in a dose dependent manner as shown in FIG. 13. 40 ng/ml of the oxSLO was sufficient for a significant signal on the standard ELISA assay. Again equal amount of unmodified sLO failed to activate HAEC. A similar result is observed in Northern blot as shown in FIG. 15.

### EXAMPLE 10

### oxSLO Induced VCAM, E-Selectin and MCP-1 Expression in HAEC

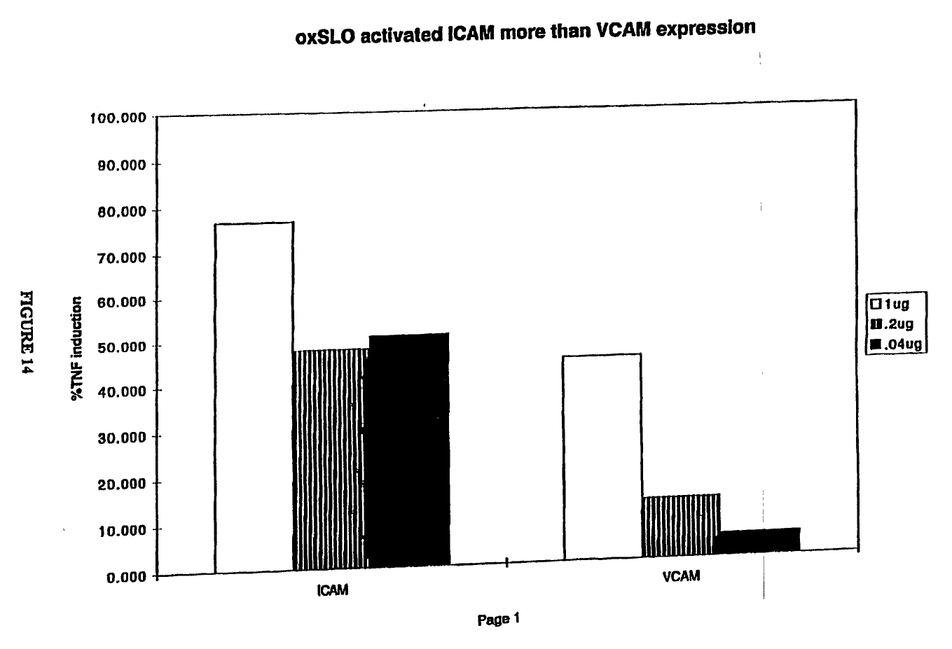

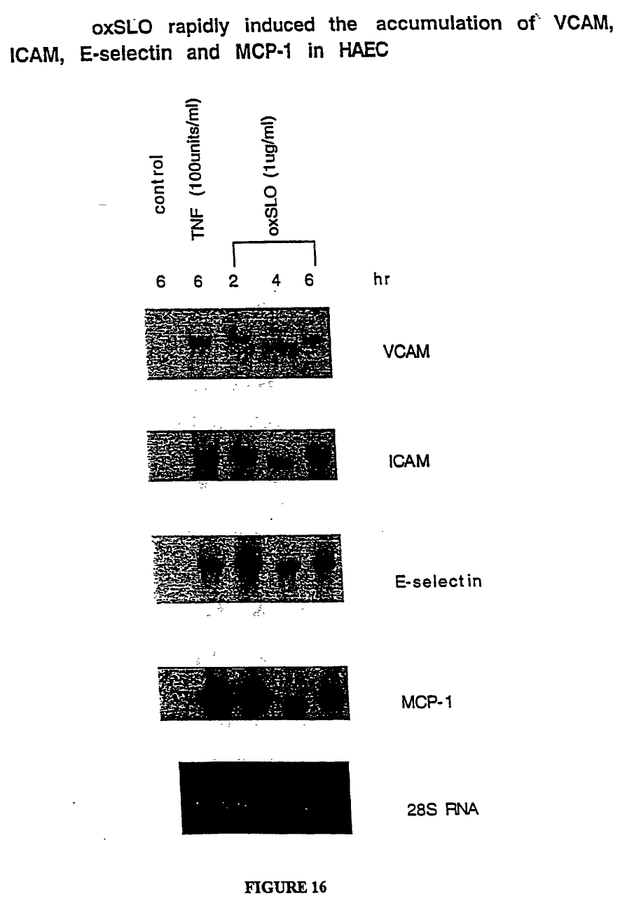

The oxSLO not only induced ICAM, but also two other adhesion molecules (VCAM in FIG. 15, E-selectin in FIG. 16) and A one chemokine molecule (MCP-1 in FIG. 15 & 16). FIG. 14 showed the induction of cell surface VCAM expression on HAEC, although to a lesser extent than the ICAM induction. The ELISA data is supported by the Northern Blot data as illustrated in FIG. 15. The amount of VCAM mRNA accumulation after 4 hr. of the oxSLO treatment was less than ICAM mRNA. Further, the MCP-1 and E-selectin mRNA level were as high as the TNF induction, suggesting that may be MCP-1 and E-selectin are key components of the oxSLO mediated signaling cascade. The induction of these proinflammatory genes are very rapid as shown in FIG. 16. Within 2-6 hr of stimulation, the mRNA level of the indicated genes all reached a steady state.

### EXAMPLE 11

oxSLO IL-15 Expression in Macrophage

Besides having a profound effect on the endothelial cells as summarized in Table 2, oxSLO induced the accumulation of another cytokine IL-1β mRNA in macrophage cell line (RAW) as shown in FIG. 14. RAW cells were treated with the oxSLO for 4 hr and total RNA were harvested and analyzed. In the development of atherosclerosis, both endothelial cells and macrophages are shown to be extremely important. Our in vitro data strongly supported the notion that a unique class of lipid hydroperoxide modified protein, lipids or lipophilic molecules which defined as oxykine can mediate a proinflammatory signal in atherosclerotic lesion areas.

TABLE 2
oxSLO induced the accumulation of VCAM-1, ICAM-1 and
MCP-1 mRNA in HAEC

| | VCAM-1 | ICAM-1 | MCP-1 |
|---|---|---|---|
| Treatment | (arbitrary units) | (arbitrary units) | (arbitrary units) |
| TNF (100 units/ml) | 82.06 | 71.92 | 101.65 |
| sLO (0.8 ug/ml) | 3.36 | 3.14 | 12.34 |
| sLO (0.08 ug/ml) | 0.12 | −0.09 | 3.1 |
| sLO (0.008 ug/ml) | −0.14 | −0.51 | 1.12 |
| modified sLO | 24.17 | 19.74 | 116.62 |
| (1 ug/ml) | | | |
| modified sLO | 11.51 | 9.41 | 96.28 |
| (0.2 ug/ml) | | | |
| modified sLO | 9.38 | 2.06 | 37.35 |
| (0.04 ug/ml) | | | |

### EXAMPLE 12

### Biological Activity of Product of Oxidative Modification of Rabbit Serum Albumin by Linoleic Hydroperoxide

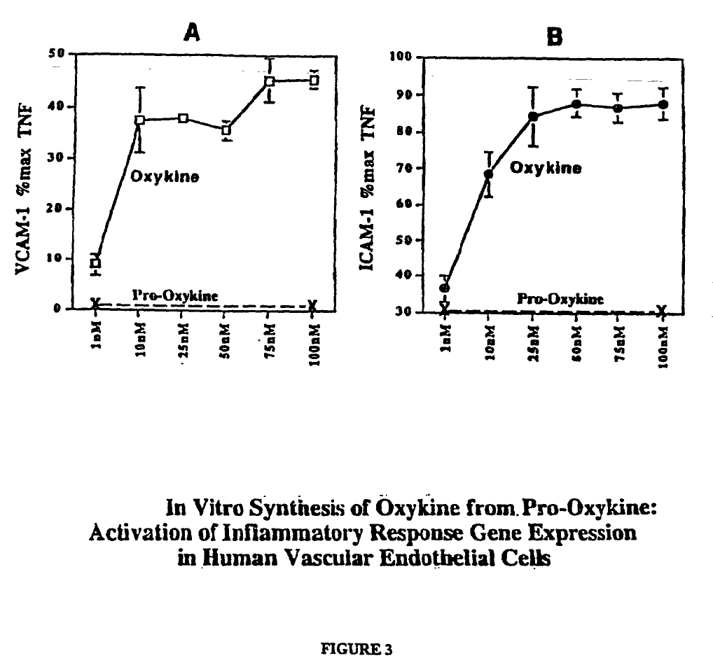

It was investigated whether the oxidative modification of rabbit serum albumin RSA by linoleic hydroperoxide alters the structural or biological property of an element (peptide or nonpeptide) in this material, conferring upon it a biological function as a vascular endothelial cell inflammatory signal. Using oxidatively modified RSA (oxRSA) prepared as described in Example 1, cultured human aortic endothelial cells were exposed for 12 hours with a range of oxRSA concentrations from 1 to 100 nM (nanomolar), and assayed for the cell surface expression of the inducible adhesion molecules VCAM-1, ICAM-1 or the constituitively expressed adhesion molecule ICAM-2 by ELISA assay. As shown in FIG. 3, expressed as a percent of the maximal TNF-a induced signal, ox-RSA exhibited a dose-dependent induction of VCAM-1 to 40% and ICAM-1 to 80% of a maximum TNF-a signal. The approximate EC₅₀ of oxRSA was 10-15 nM for both VCAM-1 and ICAM-1. This induction was not due to contaminating lipopolysaccharide as polymyxin B (10 ug/ml) fully inhibited LPS induction but had no effect on oxRSA activation. Furthermore, limulus assay for LPS in the oxRSA preparation was below detection. Constituitively expressed ICAM-2 was unaffected. This result suggests that oxRSA functions in a dose responsive manner as a potent, inflammatory factor for endothelial cells in the low nanomolar range.

### EXAMPLE 13

### Effect of Product of Oxidative Modification of Rabbit Serum Albumin by Linoleic Hydroperoxide on mRNA Accumulation

It was next investigated whether the induction of VCAM-1 and ICAM-1 cell surface expression by ox-RSA is due at least in part to changes in mRNA accumulation. Northern filter analysis was performed from RNA isolated from HAEC exposed for 4 hours With either TNFa (100 U/ml) or oxRSA (25 nM). Similar to that observed at the protein level, VCAM-mRNA levels were induced by ox-RSA to levels approximately 30-40% observed for TNFa. ICAM-1 mRNA was also induced by ox-RSA.

### EXAMPLE 14

### Effect of Product of Oxidative Modification of Rabbit Serum Albumin by Linoleic Hydroperoxide on the Transcriptional Activation of the VCAM-12 Promoter through an NF-kB Like DNA Binding Complex

To determine whether oxRSA modulated nuclear regulatory events associated with redox sensitive VCAM-1 gene expression, cultured human aortic endothelial cells were exposed or not (CTL) for two hours to TNFa (100 U/ml) or 25 nM of the oxykine RSA-LOOH (oxRSA), nuclear extracts prepared and gel mobility shift assays performed using 32P-labeled kL0kR, the tandem NF-kB-like binding sites of the human VCAM-1 promoter. Appropriate cold competitor and supershift controls using anti-p50 and anti-p65 antisera (R&D Systems) were performed to establish the DNA sequence binding specificity of the NF-kB complex band. Both TNF and oxRSA inducted NF-kB like DNA binding activity. This suggests that oxykines may play a role in NF-kB mediated transcriptional activation pathways in the vasculature.

### EXAMPLE 15

### Autoantibody Titers of Plasma Samples from Endometriosis and Control Subjects

Oxidation occurring in the peritoneal cavity of endometriosis subjects may result in presence of autoantibodies to modified proteins and such antibodies may be increased in patients suffering from endometriosis. To determine whether these autoantibodies are present in patients diagnosed with endometriosis, plasma samples taken from such patients were evaluated for the presence of antibodies that react with three antigens: the reaction product of lipid hydroperoxide with rabbit serum albumin, the reaction product of lipid hydroperoxide with LDL, and that with MDA.

ELISA assay: 10 ug of Ox-LDL, MDA-LDL or lipid peroxide-modified albumin (LOOH-RSA)positive antigens) and LDL, human albumin and acetyl LDL (negative controls) were plated in 100 ul volume in a 96 well microtiter plate. After overnight incubation at 4° C., plates are blocked by incubation with 3t milk powder by incubation for 2 hrs. Following washings bound human IfF are detected using a peroxidase or alkaline phosphatase-conjugated anti-human IgG antibody. The bound enzyme-conjugated IgG is quantitized by color detection using specific substrates.

The results are provided in Tables 3-5. As indicated, there was a statistical increase in levels of reactive antibodies in patients with endometriosis over the control.

TABLE 3
Antigen: LOOH/RSA (200 μl plasma samples). Results
are express in OD equivalents of p-nitrophenol
formed.

| | Endometriosis | Control |
|---|---|---|
| Mean | 0.493885 | 0.196313 |
| Variance | 0.117233 | 0.023264 |
| Observations | 63 | 32 |
| P (T <= t) one-tail | 3.74E-08 | |
| P (T <= t) two-tail | 7.47E-08 | |

t-Test: Two-Sample Assuming Unequal Variances

TABLE 4
Antigen: Ox-LDL (200 μl) plasma samples)

| | Endometriosis | Control |
|---|---|---|
| Mean | 0.214741935 | 0.18009375 |
| Variance | 0.005239014 | 0.006001378 |
| Observations | 62 | 32 |
| P (T <= t) one-tail | 0.019975579 | |
| P (T <= t) two-tail | 0.039951158 | |

t-Test: Two-Sample Assuming Unequal Variances

TABLE 5
Antigen: MDA-LDL (200 μl plasma samples)

| | Endometriosis | Control |
|---|---|---|
| Mean | 0.213758065 | 0.165406 |
| Variance | 0.005736645 | 0.003401 |
| Observations | 62 | 32 |
| P (T <= t) one-tail | 0.000485039 | |
| P (T <= t) two-tail | 0.000970078 | |

t-Test: Two-Sample Assuming Unequal Variances

This invention has been described with reference to its preferred embodiments. Variations and modifications of the method, kit, and materials, including antibodies, will be obvious to those skilled in the art from the foregoing detailed description of the invention. It is intended that all of these variations and modifications be included within the scope of the appended claims.

TABLE 1
oxSLO induced the accumulation of VCAM-1, ICAM-1 and MCP-1
mRNA in HAEC

| | VCAM-1 | ICAM-1 | MCP-1 |
|---|---|---|---|
| | (arbitrary | (arbitrary | (arbitrary |
| Treatment | units) | units) | units) |
| TNF (100 units/ml) | 82.06 | 71.92 | 101.65 |
| sLO (0.8 ug/ml) | 3.36 | 3.14 | 12.34 |
| sLO (0.08 ug/ml) | 0.12 | −0.09 | 3.1 |
| sLO (0.008 ug/ml) | −0.14 | −0.51 | 1.12 |
| modified sLO (1 ug/ml) | 24.17 | 19.74 | 116.62 |
| modified sLO (0.2 ug/ml) | 11.51 | 9.41 | 96.28 |
| modified sLO (0.04 ug/ml) | 9.38 | 2.06 | 37.35 |

We claim:

1. A method for the assessment of lipid peroxidation in a biological sample that includes contacting the biological sample with an antibody which binds to an antigen formed by the reaction of a lipid hydroperoxide with a primary amine.

2. A kit for the assessment of lipid peroxidation in a biological sample that includes (I) an antibody or antibody fragment that binds to a reaction product of a lipid hydroperoxide with a primary amine, or (ii) an antibody which binds to the antibody of (I).

3. A isolated antibody which binds to an antigen formed by reacting a lipid hydroperoxide with a primary amine.

4. An antibody fragment which binds to an antigen formed by reacting a lipid hydroperoxide with a primary amine.

5. The antibody of claim 3 or 4, immobilized on a solid support.

6. The antibody of claim 3 or 4, labeled with a detectable agent.

7. The antibody of claim 5, wherein the solid support is selected from a membrane and a coating supported by or attached to sticks, beads, cups, or flat packs.

8. The antibody of claim 5, wherein the solid support is selected from a cell culture plate, ELISA plate, tube, and polymeric membrane.

9. The antibody of claim 3 or 4, that is labeled with a detectable agent selected from the group consisting of a fluorochrome, a radioactive label, biotin, horseradish peroxidase, alkaline phosphatase, 2-galactosidase, or other enzyme

10. The antibody of claim 3, which is a conjugate.

11. The antibody of claim 3 or 4, wherein the antigen is the reaction product of linoleic hydroperoxide and a primary amine.

12. The antibody of claim 11, wherein the appropriate amino acid group, such as lysine, in albumin or polylysine, or a small molecular weight compound such as phosphatidylethanolamine.

13. The antibody of claim 3 or 4 which is humanized.

14. A method for the assessment of the level of lipid peroxidation in a biological sample, that includes contacting the sample with either: (I) an antigen which binds to an antibody which is immunoreactive with an antigen formed by the reaction of a lipid hydroperoxide with a primary amine or (ii) an antibody or antibody fragment or conjugate that is crossreactive with an antibody that is immunoreactive with an antigen formed by the reaction of a lipid hydroperoxide with a primary amine.

14. The method of claim 13, wherein the level of (I) or (ii) in a host is compared to a population norm.

15. A method for assessing oxidative damage in a biological sample, comprising the steps of:
(I) isolating an antigen formed by the reaction of a lipid hydroperoxide with a primary amine; and then
(ii) identifying the primary amine.

16. A method for diagnosing an oxidation-related disorder in a biological sample, comprising assessing the level of a reaction product of a lipid hydroperoxide with a primary amine.

17. The method of claim 6, wherein the oxidation related condition is selected from the group consisting of cardiovascular disease, atherosclerosis, inflammatory disease, endometriosis, glymerol nephritis, preeclampsia, central nervous system disorders mediated by lipid peroxidation, Alzheimer's disease, psoriasis, asthma, atopic dermatitis, solid tumors, Kaposi's sarcoma, neurodegenerative disease, inflammatory bowel disease (Crohn's disease), rheumatoid arthritis, and ischemia reperfusion.

18. A method for inducing an inflammatory effect in a biological sample or host, that includes contacting the sample or host with a fluorescent product of the reaction between a lipid hydroperoxide and a primary amine which induces such an effect.

19. The method of claim 13, wherein the linoleic hydroperoxide is 13-HPODE.

20. The method of claim 13, wherein the amine is selected form the group consisting of an amino acid, protein, peptide, or phosphatidylethanolamine.

21. The method of claim 131 wherein the inflammatory effect is produced by induction of a mediator selected from the group consisting of VCAM-1, MCP-1, IL-1, TNF-α, ICAM, MCSF, and E-selectin.

22. The antibody of claim 6, in combination with a means for detecting the detectable agent.

23. The antibody of claim 21, wherein the combined means for detecting a detectable agent employs an enzyme as a detectable agent and an enzyme substrate that changes color upon contact with the enzyme.

24. A method for evaluating the ability of a substance to lower the state of lipid peroxidation of a biological sample comprising comparing the level of the reaction product of a lipid hydroperoxide and a primary amine in the host sample before and after contacting the sample with the substance.


SEQUENCE LISTING (1 sequence)

SEQ ID NO: 1 - protein, 26 aa, Artificial Sequence
  Description of Artificial Sequence SYNTHETIC SEQUENCE
YVTKSYNETKIKFDKYKAEKSHEDEL